SEQUENCE LISTING (26 sequences)

SEQ ID NO: 1 - DNA, 24 bp, Artificial Sequence
  Primer used for RT-PCR
tgcagccaaagtgaagagggaaga

SEQ ID NO: 2 - DNA, 24 bp, Artificial Sequence
  Primer used for RT-PCR
catagcgagcagcccaaagaagaa

SEQ ID NO: 3 - DNA, 21 bp, Artificial Sequence
  Primer used for RT-PCR
gaccagatctcccttctcaag

SEQ ID NO: 4 - DNA, 21 bp, Artificial Sequence
  Primer used for RT-PCR
ctcaggctcttggagctgcag

SEQ ID NO: 5 - DNA, 20 bp, Artificial Sequence
  Primer used for RT-PCR
atggccgagcagaaccggaa

SEQ ID NO: 6 - DNA, 20 bp, Artificial Sequence
  Primer used for RT-PCR
ccatgagccgctggtactcc

SEQ ID NO: 7 - DNA, 21 bp, Artificial Sequence
  Primer used for RT-PCR
gacgctacgtttcagtctttc

SEQ ID NO: 8 - DNA, 19 bp, Artificial Sequence
  Primer used for RT-PCR
gctgaataccacgccatag

SEQ ID NO: 9 - DNA, 22 bp, Artificial Sequence
  Primer used for RT-PCR
ttcctaaggacttctgctttgc

SEQ ID NO: 10 - DNA, 24 bp, Artificial Sequence
  Primer used for RT-PCR
tgtggaggaaattattgagaaatg

SEQ ID NO: 11 - DNA, 18 bp, Artificial Sequence
  Primer used for RT-PCR
accagtggatgcagggat

SEQ ID NO: 12 - DNA, 18 bp, Artificial Sequence
  Primer used for RT-PCR
tcaacggcacagtgaagg

SEQ ID NO: 13 - DNA, 20 bp, Artificial Sequence
  Primer used for RT-PCR
tattggctgcagctaagcgg

SEQ ID NO: 14 - DNA, 20 bp, Artificial Sequence
  Primer used for RT-PCR
gactcggactcaggtgaggt

SEQ ID NO: 15 - DNA, 20 bp, Artificial Sequence
  Primer used for RT-PCR
ccaagtacatcccagctttc

SEQ ID NO: 16 - DNA, 18 bp, Artificial Sequence
  Primer used for RT-PCR
ttggcatctgggtcaaag

SEQ ID NO: 17 - DNA, 19 bp, Artificial Sequence
  Primer used for RT-PCR
tctgccaaggtcatccagg

SEQ ID NO: 18 - DNA, 19 bp, Artificial Sequence
  Primer used for RT-PCR
gttttggggatgggcactg

SEQ ID NO: 19 - DNA, 20 bp, Artificial Sequence
  Primer used for RT-PCR
ggcacgcagtcctattcatt

SEQ ID NO: 20 - DNA, 19 bp, Artificial Sequence
  Primer used for RT-PCR
acaggggagaatttcggtg

SEQ ID NO: 21 - DNA, 20 bp, Artificial Sequence
  Primer used for RT-PCR
agaccactcccactcctttg

SEQ ID NO: 22 - DNA, 20 bp, Artificial Sequence
  Primer used for RT-PCR
aggtcatacttgtaatctgc

SEQ ID NO: 23 - DNA, 21 bp, Artificial Sequence
  Primer used for RT-PCR
caagcggaagaaaagtgaacg

SEQ ID NO: 24 - DNA, 21 bp, Artificial Sequence
  Primer used for RT-PCR
ctggtcctcgatgggcaagtc

SEQ ID NO: 25 - DNA, 20 bp, Artificial Sequence
  Primer used for RT-PCR
tgaactggctgactgctgtg

SEQ ID NO: 26 - DNA, 20 bp, Artificial Sequence
  Primer used for RT-PCR
catccttggcctcagcatag

(19) United States
(12) Patent Application Publication
(10) Pub. No.: US 2008/0019949 A1
(43) Pub. Date: Jan. 24, 2008

(54) DIFFERENTIATION OF STEM CELLS FROM UMBILICAL CORD MATRIX INTO HEPATOCYTE LINEAGE CELLS

(75) Inventors: Kathy E. Mitchell, Lawrence, KS (US); Steven M. Hoynowski, Eudora, KS (US)

Correspondence Address: SEED INTELLECTUAL PROPERTY LAW GROUP PLLC, 701 FIFTH AVE, SUITE 5400, SEATTLE, WA, 98104, US

(73) Assignee: UNIVERSITY OF KANSAS, Lawrence, KS (US)

(21) Appl. No.: 11/770,544

(22) Filed: Jun. 28, 2007

Related U.S. Application Data
(60) Provisional application No. 60/817,251, filed on Jun. 28, 2006.

(51) Int. Cl. C12Q 1/02; A61K 35/407; C12M 1/34; C12Q 1/68; C12N 5/06

(52) U.S. Cl. 424/93.7; 435/288.4; 435/29; 435/377; 435/6

(57) ABSTRACT

The invention relates to methods for differentiating umbilical cord matrix cells into hepatocyte-like cells and compositions and methods for using such hepatocyte-like cells.

### CROSS-REFERENCE TO RELATED APPLICATION

This application claims the benefit under 35 U.S.C. § 119(e) of U.S. Provisional Patent Application No. 60/817,251, filed Jun. 28, 2006, where this provisional application is incorporated herein by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 120157_{—}418_SEQUENCE_LISTING.txt. The text file is 5 KB, was created on Jun. 28, 2007, and is being submitted electronically via EFS-Web, concurrent with the filing of the specification.

### BACKGROUND OF THE INVENTION

1. Field of the Invention

The invention relates to the isolation and use of stem cells from any animal with an umbilical cord, including humans, for differentiation into cells of the hepatocyte lineage. More particularly the invention relates to methods for differentiating umbilical cord matrix cells into hepatocyte-like cells. The invention is also useful for providing a readily available supply of hepatocyte-like cells for use in a variety of settings including drug screening, drug-drug interaction, transplantation and disease treatment.

2. Description of the Related Art

Treatment of liver disease by organ transplantation has shown efficacy and progress. However, the problem with transplantation regimes have been centered on organ availability and suitability. The lack of suitable organ donors has been increasing and the need for alternate therapies exists. Cell based therapies have shown some promise in the regenerative medicine field but lack the efficacy of transplantation. More research needs to be accomplished in this area the fully develop cell-based therapies for the treatment of liver disease (Allen J W and Bhatia S N. Tissue Engineering. 2002; 8(5):725-737; H. C. Fiegel C L, et al. J Cell Mol. Med. 2006; 10(3):577-587).

Induction and inhibition of Cytochrome P450s are a key mechanism for the oxidative metabolism of drugs and other xenobiotics. Hepatic models to study drug metabolism in humans is of clinical interest. Primary cultures of hepatocytes do express drug-metabolizing activities for a time period but lose this ability in long term culture. Other obstacles for using primary hepatocyte cultures include: ethical reasons, availability of tissue from donors, short useable lifespan of primary cultures (Donato M, et al. Drug Metab Dispos. 1995; 23(5):553-558; Li A P, et al. Chemico-Biological Interactions Proceedings of the First Symposium of the Hepatocyte Users Group of North America. 1997; 107(1-2):5-16). Therefore, a suitable model for studying drug interactions and cytotoxicity would prove to be advantageous in screening new drugs, or new therapeutic products.

The liver is a major site of metabolism of many endogenous compounds and xenobiotics since hepatocytes (which comprise 80% of the liver cells) contain large amounts of smooth endoplasmic reticulum, where many metabolizing enzymes reside. These metabolizing enzymes are primarily involved in two major types of processes: redox reactions catalyzed by P450 monooxygenases (phase I) and conjugation with endogenous molecules (phase II). Much effort in drug discovery and development has focused on defining the metabolic profile and the pharmacokinetics of new compounds. A major portion of preclinical development involves characterizing the liver enzymes affecting drug disposition and elimination.

Over thirty drugs have been associated with severe, often fatal, drug toxicity which was realized only after marketing. One limitation of current technologies for early testing of drug toxicity is the lack of genetic diversity of the testing systems. Thus, there remains a need in the art for a readily available and genetically diverse supply of hepatocyte-like cell lines for early drug toxicity testing. The present invention provides this and other advantages.

### BRIEF SUMMARY OF THE INVENTION

One aspect of the invention provides a method for differentiating umbilical cord matrix cells into hepatocyte-like cells, comprising contacting umbilical cord matrix cells with Pre-induction Media; contacting umbilical cord matrix cells with Differentiation Media; and contacting umbilical cord matrix cells with Maturation Media, for a time sufficient to differentiate the umbilical cord matrix cells into hepatocyte-like cells.

Another aspect of the invention provides a method for evaluating the toxicity of a compound in vitro, comprising contacting a hepatocyte-like cell differentiated from umbilical cord matrix cells according to the invention with said compound; and measuring the viability of said hepatocyte-like cell, wherein a decrease in viability in the presence of said compound compared to that in the absence of said compound indicates that said compound is toxic in vivo.

A further aspect of the invention provides a method for evaluating the activity of a compound in vitro, comprising contacting a metabolically active hepatocyte-like cell differentiated from umbilical cord matrix cells according to the invention with said compound; and measuring the metabolic activity of said hepatocyte-like cell, wherein a decrease or increase in metabolic activity in the presence of said compound compared to that in the absence of said compound indicates that said compound has activity in vivo.

Yet a further aspect of the invention provides a method for evaluating the activity of a compound in vitro, comprising contacting a first metabolically active hepatocyte-like cell differentiated from umbilical cord matrix cells according to the invention with said compound to generate a cell supernatant; and contacting a second metabolically active hepatocyte-like cell differentiated from umbilical cord matrix cells according to the invention with said supernatant; and measuring the metabolic activity of said second hepatocyte-like cell, wherein a decrease or increase in metabolic activity in the presence of said supernatant compared to that in the absence of said supernatant indicates that said compound has activity in vivo.

An additional aspect of the invention is a method for evaluating the toxicity of a compound in vitro, comprising contacting a first metabolically-active hepatocyte-like cell differentiated from umbilical cord matrix cells according to the invention with said compound to generate a cell supernatant; contacting a second metabolically-active hepatocyte-like cell differentiated from umbilical cord matrix cells according to the invention with said cell supernatant; and measuring the viability of said second hepatocyte-like cell, wherein a decrease in viability in the presence of said supernatant compared to that in the absence of said supernatant indicates that said compound is toxic in vivo.

Another aspect of the invention provides a method for evaluating the activity of a compound in vitro, comprising contacting a hepatocyte-like cell differentiated from umbilical cord matrix cells according to the invention with said compound; and measuring the expression of a cytochrome P450 gene in the hepatocyte-like cell, wherein an increase or decrease in expression of the cytochrome P450 gene in the presence of said compound compared to that in the absence of said compound indicates that said compound has actvity in vivo.

An additional aspect of the invention provides a method for evaluating the activity of a compound in vitro, comprising contacting a first metabolically active hepatocyte-like cell differentiated from umbilical cord matrix cells according to the invention with said compound to generate a cell supernatant; and contacting a second metabolically active hepatocyte-like cell differentiated from umbilical cord matrix cells according to the invention with said supernatant; and measuring expression of a cytochrome P450 gene in said second hepatocyte-like cell, wherein an increase or decrease in expression of the cytochrome P450 gene in the presence of said supernatant compared to that in the absence of said supernatant indicates that said compound has activity in vivo. In one embodiment, the cytochrome P450 gene expression is measured using the polymerase chain reaction. In a further embodiment the cytochrome P450 gene expression is measured by measuring enzyme activity.

Another aspect of the invention provides a method for determining drug interactions, comprising contacting a first hepatocyte-like cell differentiated from umbilical cord matrix cells according to the invention with a first compound; contacting a second hepatocyte-like cell differentiated from umbilical cord matrix cells according to the invention with a second compound; contacting a third hepatocyte-like cell differentiated from umbilical cord matrix cells according to the invention with the first and the second compound; measuring the metabolic activity of the first, second and third hepatocyte-like cell, wherein a decrease or increase in metabolic activity in the third hepatocyte-like cell as compared to the first or the second hepatocyte-like cell or both indicates a drug interaction.

A further aspect of the invention provides a method for determining drug interactions, comprising: contacting a first hepatocyte-like cell differentiated from umbilical cord matrix cells according to the invention with a first compound; contacting a second hepatocyte-like cell differentiated from umbilical cord matrix cells according to the invention with a second compound; contacting a third hepatocyte-like cell differentiated from umbilical cord matrix cells according to the invention with the first and the second compound; measuring the viability of the first, second and third hepatocyte-like cells, wherein a decrease or increase in viability in the third hepatocyte-like cell as compared to the first or the second hepatocyte-like cell or both indicates a drug interaction.

Yet a further aspect of the invention provides a method for determining drug interactions, comprising: contacting a first hepatocyte-like cell differentiated from umbilical cord matrix cells according to the invention with a first compound; contacting a second hepatocyte-like cell differentiated from umbilical cord matrix cells according to the invention with a second compound; contacting a third hepatocyte-like cell differentiated from umbilical cord matrix cells according to the invention with the first and the second compound; measuring the expression of a cytochrome P450 gene in the first, second and third hepatocyte-like cells, wherein a decrease or increase in the expression of a cytochrome P450 gene in the third hepatocyte-like cell as compared to the first or the second hepatocyte-like cell or both indicates a drug interaction.

Another aspect of the invention provides a method for improving or restoring liver function in an individual in need thereof comprising administering to the individual in need thereof a population of hepatocyte-like cells differentiated from umbilical cord matrix cells according to the invention.

A further aspect of the invention provides a method for treating cirrhosis of the liver in an individual in need thereof comprising administering to the individual a population of hepatocyte-like cells differentiated from umbilical cord matrix cells according to the invention.

Yet another aspect of the invention provides a method for treating liver damage comprising administering to an individual who has sustained liver damage a population of hepatocyte-like cells differentiated from umbilical cord matrix cells according to the invention.

Yet another aspect of the invention provides a method for treating hepatitis comprising administering to an individual who has sustained liver damage a population of hepatocyte-like cells differentiated from umbilical cord matrix cells according to the invention.

Another aspect of the invention provides a panel of umbilical cord matrix-derived hepatocyte-like cells comprising at least two umbilical cord matrix-derived hepatocyte-like cells wherein the at least two umbilical cord matrix-derived hepatocyte-like cells are derived from different subjects, and wherein the umbilical cord matrix-derived hepatocyte-like cells are separate one from the other. Thus, the cells are provided in distinct, separate locations on the panel. In one embodiment, the different subjects are genetically different. In another embodiment, the different subjects are of different sexes. Thus a panel may be comprised of cells derived from umbilical cords of female and male subjects. In one embodiment, the at least two umbilical cord matrix-derived hepatocyte-like cells are separated one from the other in a multi-well plate. In a further embodiment, the panel comprises at least three, four, five, six, seven, eight, nine, ten, or more different umbilical cord matrix-derived hepatocyte-like cells. In this regard, the panels of the invention may comprise between 5 and 100 or more different umbilical cord matrix-derived hepatocyte-like cells, all provided in separate locations, such as in a multiwell tissue culture plate.

A further aspect of the invention provides a drug screening kit comprising a panel of the invention and at least one reagent for measuring at least one cytochrome P450 enzyme activity or gene expression. In one embodiment, the kit comprises at least one medium for culturing the umbilical cord matrix-derived hepatocyte-like cells.

Another aspect of the invention provides a method for differentiating umbilical cord matrix cells into hepatocyte-like cells, comprising: seeding umbilical cord matrix cells on a 0.1% gelatin coated tissue culture plate; contacting umbilical cord matrix cells with a Pre-induction Media comprising 10-30 ng/ml recombinant human epidermal growth factor and 5-15 ng/ml recombinant human basic fibriblast growth factor; contacting umbilical cord matrix cells with a Differentiation Media comprising 10-30 ng/ml recombinant human hepatocyte growth factor, 5-15 ng/ml rhbFGF and 0.5-1.0 g/L nicotinamide; and contacting umbilical cord matrix cells with a Maturation Media comprising 10-30 ng/ml Human Oncostatin M, 0.5-1.5 umol/L dexamethasone and 30-70 mg/ml ITS+ premix; for a time sufficient to differentiate the umbilical cord matrix cells into hepatocyte-like cells.

### DETAILED DESCRIPTION OF THE INVENTION

The present invention relates generally to methods for differentiating umbilical cord matrix stem cells into cells of the hepatocyte lineage and compositions comprising and methods of using the cells.

Multiple studies have demonstrated the usefulness of extra-embryonic tissues were these components can differentiate into hepatocyte-like cells in vitro (Lee O K, et al. Blood. 2004; 103(5):1669-1675; Schwartz R E, et al. J Clin Invest. 2002; 109(10):1291-1302; Hong S H, et al. Biochemical and Biophysical Research Communications. 2005; 330(4):1153-1161; Sato Y, et al. Blood. 2005; 106(2):756-763). Hepatic differentiation protocols accomplish differentiation with a monolayer of progenitor cells that are treated with various growth factors to induce differentiation (Ong S—Y, Dai H, Leong K W. Tissue Engineering. 2006; 12(12):3477-3485; Lee O K, et al. Blood. 2004; 103(5):1669-1675; Schwartz R E, et al. J Clin Invest. 2002; 109(10):1291-1302; Hong S H, et al. Biochemical and Biophysical Research Communications. 2005; 330(4):1153-1161; Yamada T, et al. Stem Cells. 2002; 20(2):146-154; Koenig S, et al. Journal of Hepatology. 2006; 44(6):1115-1124; Chien C-C, et al. Stem Cells. 2006; 24(7):1759-1768; Forte G, et al. Stem Cells. 2006; 24(1):23-33). It has been shown that primary hepatocytes sustain viability in long-term culture conditions, maintain liver-specific functions, and have structural similarities to native liver tissue when cultured in a more supportive three-dimensional (3D) system. In two-dimensional (2D) culture systems, hepatocytes lose their polarity, which is important for trafficking metabolites, and hinder development canalicular or sinusoidal structures (Hamamoto R, et al. J Biochem (Tokyo). 1998; 124(5):972-979; Landry J, et al. J. Cell Biol. 1985; 101(3):914-923; Abu-Absi S F, Friend J R, et al. Experimental Cell Research. 2002; 274(1):56-67). In addition, ECM (extra cellular matrix) plays a physiological role by influencing the microenvironment of hepatocytes where organism-compatible materials combined with extracellular matrices are capable of promoting cell differentiation (HENG BC, et al. Journal of Gastroenterology and Hepatology. 2005; 20(7):975-987).

When considering that a large amount of functional cells would be required for drug discovery and toxicity studies, a scalable, economic model would be required. The present invention provides cells of the hepatocyte lineage differentiated from human umbilical cord-derived matrix cells which can be used in a variety of settings, including induction of relevant cytochrome P450s for drug testing. The present invention provides an additional source for cell-based drug therapies, toxicity studies, and a possible source of cells for transplantation in certain pathologies.

Isolation and Culture of Umbilical Cord Matrix (UCM) cells.

Stem cells are capable of self-regeneration and can become lineage committed progenitors which are dedicated to differentiation and expansion into a specific lineage.

Following fertilization of an egg by a sperm, a single cell is created that has the potential to form an entire differentiated multi-cellular organism including every differentiated cell type and tissue found in the body. This initial fertilized cell, with total potential is characterized as totipotent. Such totipotent cells have the capacity to differentiate into extra-embryonic membranes and tissues, embryonic tissues and organs. After several cycles (5 to 7 in most species) of cell division, these totipotent cells begin to specialize forming a hollow sphere of cells, the blastocyst. The inner cell mass of the blastocyst is composed of stem cells described as pluripotent because they can give rise to many types of cells that will constitute most of the tissues of an organism (not including some placental tissues etc.). Multipotent stem cells are more specialized giving rise to a succession of mature functional cells. The multipotent stem cell can give rise to hematopoietic, mesenchymal or neuroectodermal cell lines. Thus, the hierarchy of stem cells is: totipotent stem cells→pluripotent stem cells→multipotent stem cell→committed cell lineage.

True pluripotent stem cells should: (i) be capable of indefinite proliferation in vitro in an undifferentiated state; (ii) maintain a normal karyotype through prolonged culture; and (iii) maintain the potential to differentiate to derivatives of all three embryonic germ layers (endoderm, mesoderm, and ectoderm) even after prolonged culture. Strong evidence of these required properties have been published only for rodent embryonic stem cells (ES cells) and embryonic germ cells (EG cells) including mouse (Evans & Kaufman, Nature 292: 154-156, 1981; Martin, Proc Natl Acad Sci USA 78: 7634-7638, 1981) hamster (Doetschman et al. Dev Biol 127: 224-227, 1988), and rat (lannaccone et al. Dev Biol 163: 288-292, 1994), and less conclusively for rabbit ES cells (Giles et al. Mol Reprod Dev 36: 130-138, 1993; Graves & Moreadith, Mol Reprod Dev 36: 424-433, 1993). However, only established stem cell lines from the rat (lannaccone, et al., 1994, supra) and the mouse (Bradley, et al., Nature 309: 255-256, 1984) have been reported to participate in normal development in chimeras.

Human pluripotent cells have been developed from two sources with methods previously developed in work with animal models. Pluripotent stem cells have been isolated directly from the inner cell mass of human embryos (ES cells) at the blastocyst stage obtained from in vitro fertilization programs. Pluripotent stem cells (EG cells) have also been isolated from terminated pregnancies.

The present invention provides umbilical cord matrix (UCM) stem cells that can be used to differentiate into cells of the hepatocyte lineage. UCM can be isolated using techniques known in the art, such as described in U.S. Pat. No. 5,919,702 and US Patent Application Publication No. 20040136967. Umbilical Cord Matrix (UCM) stem cells are also known as Wharton's Jelly Cells. Such cells can be found in nearly any animal with an umbilical cord, including amniotes, placental animals, humans, and the like. Such matrix cells typically include extravascular cells, mucous-connective tissue (e.g., Wharton's Jelly) but typically do not include cord blood cells or related cells. Any of these cells may provide a source for differentiated cells and can provide an important feeder environment for the establishment or maintenance of stem cell cultures. UCM stem cells derived from umbilical cord tissue can be isolated, purified and culturally expanded.

UCM cells are isolated from a non-blood tissue specimen from umbilical cord containing UCM cells. The UCM cells are then added to a medium which contains factors that stimulate UCM cell growth without differentiation and allows, when cultured, for the selective adherence of the UCM stem cells to a substrate surface. The specimen-medium mixture is cultured and the non-adherent matter is removed from the substrate surface. The use of umbilical cord blood is also discussed, for instance, in Issaragrishi et al. (1995) N. Engl. J. Med. 332:367-369.

The UCM stem cells of the invention are isolated from umbilical cord sources, preferably from Wharton's jelly. Wharton's jelly is a gelatinous substance found in the umbilical cord which has been generally regarded as a loose mucous connective tissue, and has been frequently described as consisting of fibroblasts, collagen fibers and an amorphous ground substance composed mainly of hyaluronic acid (Takechi et al., 1993, Placenta 14:235-45). Various studies have been carried out on the composition and organization of Wharton's jelly (Gill and Jarjoura, 1993, J. Rep. Med. 38:611-614; Meyer et al., 1983, Biochim. Biophys. Acta 755:376-387). One report described the isolation and in vitro culture of “fibroblast-like” cells from Wharton's jelly (McElreavey et al., 1991, Biochem. Soc. Trans. 636th Meeting Dublin 19:29 S).

Umbilical cord is generally obtained immediately upon termination of either a full term or pre-term pregnancy. For example, but not by way of limitation, the umbilical cord, or a section thereof, may be transported from the birth site to the laboratory in a sterile container such as a flask, beaker or culture dish, containing a medium, such as, for example, Dulbecco's Modified Eagle's Medium (DMEM). The umbilical cord is preferably maintained and handled under sterile conditions prior to and during collection of the Wharton's jelly, and may additionally be surface-sterilized by brief surface treatment of the cord with, for example, a 70% ethanol solution, followed by a rinse with sterile, distilled water. The umbilical cord can be briefly stored for up to about three hours at about 3-5° C., but not frozen, prior to extraction of the Wharton's jelly.

Wharton's Jelly is collected from the umbilical cord under sterile conditions by an appropriate method known in the art. For example, the cord is cut transversely with a scalpel, for example, into approximately one inch sections, and each section is transferred to a sterile container containing a sufficient volume of phosphate buffered saline (PBS) containing CaCl₂ (0.1 g/l) and MgCl₂6H₂O (0.1 g/l) to allow surface blood to be removed from the section by gentle agitation. The section is then removed to a sterile-surface where the outer layer of the section is sliced open along the cord's longitudinal axis. The blood vessels of the umbilical cord (two veins and an artery) are dissected away, for example, with sterile forceps and dissecting scissors, and the umbilical cord is collected and placed in a sterile container, such as a 100 mm TC-treated Petri dish. The umbilical cord may then be cut into smaller sections, such as 2-3 mm³ for culturing. Another method relies on enzymatic dispersion of Wharton's Jelly with collagenase and isolation of cells by centrifugation followed by plating.

Wharton's jelly is incubated in vitro in culture medium under appropriate conditions to permit the proliferation of any UCM cells present therein. Any appropriate type of culture medium can be used to isolate the UCM cells of the invention, such as, but not limited to, DMEM, McCoys 5A medium (Gibco), Eagle's basal medium, CMRL medium, Glasgow minimum essential medium, Ham's F-12 medium, Iscove's modified Dulbecco's medium, Liebovitz' L-15 medium, and RPMI 1640, among others. The culture medium may be supplemented with one or more components including, for example, fetal bovine serum (FBS), equine serum (ES), human serum (HS), and one or more antibiotics and/or antimycotics to control microbial contamination, such as, for example, penicillin G, streptomycin sulfate, amphotericin B, gentamicin, and nystatin, either alone or in combination, among others.

Methods for the selection of the most appropriate culture medium, medium preparation, and cell culture techniques are well known in the art and are described in a variety of sources, including Doyle et al., (eds.), 1995, Cell and Tissue Culture Laboratory Procedures, John Wiley & Sons, Chichester; and Ho and Wang (eds.), 1991, Animal Cell Bioreactors, Butterworth-Heinemann, Boston, which are incorporated herein by reference.

Culturing UCM cells involves fractionating the source of cells (Wharton's Jelly) into two fractions, one of which is enriched for stem cells and thereafter exposing the stem cells to conditions suitable for cell proliferation. The cell enriched isolate thus created comprises stem cells.

After culturing Wharton's Jelly for a sufficient period of time, for example, about 10-12 days, UCM derived stem cells present in the explanted tissue will tend to have grown out from the tissue, either as a result of migration therefrom or cell division or both. These UCM derived stem cells may then be removed to a separate culture vessel containing fresh medium of the same or a different type as that used initially, where the population of UCM derived stem cells can be mitotically expanded.

Alternatively, the different cell types present in Wharton's Jelly can be fractionated into subpopulations from which UCM derived stem cells can be isolated. This may be accomplished using standard techniques for cell separation including, but not limited to, enzymatic treatment to dissociate Wharton's Jelly into its component cells, followed by cloning and selection of specific cell types (for example, myofibroblasts, stem cells, etc.), using either morphological or biochemical markers, selective destruction of unwanted cells (negative selection), separation based upon differential cell agglutinability in the mixed population as, for example, with soybean agglutinin, freeze-thaw procedures, differential adherence properties of the cells in the mixed population, filtration, conventional and zonal centrifugation, centrifugal elutriation (counter-streaming centrifugation), unit gravity separation, countercurrent distribution, electrophoresis, and fluorescence activated cell sorting (FACS). For a review of clonal selection and cell separation techniques, see Freshney, 1994, Culture of Animal Cells; A Manual of Basic Techniques, 3d Ed., Wiley-Liss, Inc., New York.

In one embodiment for culturing UCM derived stem cells, Wharton's Jelly is cut into sections, such as section of approximately 1-5 mm³, and placed in an appropriate dish, such as a TC-treated Petri dish containing glass slides on the bottom of the Petri dish. The tissue sections are then covered with another glass slide and cultured in a complete medium, such as, for example, Dulbecco's MEM plus 20% FBS; or RPMI 1640 containing 10% FBS, 5% ES and antimicrobial compounds, including penicillin G (100 ug/ml), streptomycin sulfate (100 ug/ml), amphotericin (250 ug/ml), and gentamicin (10 ug/ml), pH 7.4-7.6. The tissue is preferably incubated at 37-39° C. and 5% CO₂ for 10-12 days. However, as would be recognized by the skilled artisan, the temperature, O₂ and CO₂ levels can be adjusted. For example the temperature may range from 32°-40° C. and the CO₂ level may range in certain embodiments from 2%-7%. The number of days in culture can also be adjusted from about 5, 6, 7, 8, or 9 to about 13, 14, 15, 20, 25 or more days. A further example of a defined media is DMEM, 40% MCDB201, 1× insulin-transferrin-selenium (ITS), 1× linoleic acid-BSA, 10⁻⁸ M dexamethasone, 10⁻⁴ M ascorbic acid 2-phosphate, 100 U penicillin, 1000 U streptomycin, 2% FBS, 10 ng/mL EGF, 10 ng/mL PDGF-BB.

The medium is changed as necessary by carefully aspirating the medium from the dish, for example, with a pipette, and replenishing with fresh medium. Incubation is continued as above until a sufficient number or density of cells accumulates in the dish and on the surfaces of the slides. For example, the culture obtains approximately 70 percent confluence but not to the point of complete confluence. The original explanted tissue sections may be removed and the remaining cells are trypsinized using standard techniques. After trypsinization, the cells are collected, removed to fresh medium and incubated as above. The medium is changed at least once at 24 hr post-trypsin to remove any floating cells. The cells remaining in culture are considered to be UCM derived stem cells.

In another embodiment, UCM cells are isolated and cultured as follows: umbilical cords are obtained from full term infants in accordance with the appropriate Human Subjects Approval. The human umbilical cord matrix (HUCM) cells are grown from umbilical cord tissue that was processed in the following manner: the cord is prepared for processing by rinsing in a 1000 mL beaker containing approximately 500 mL of 95% ethanol or sufficient amount to completely cover the cord, for 30 seconds. The cord is then flamed until the ethanol is dissipated, then washed thoroughly 2×, for 5 minutes, in cold sterile PBS (500 mL). Next, the cord is submerged in 500 mL Betadine solution 1× for 5 minutes followed by rinsing thoroughly 2× for 5 minutes with cold sterile PBS (500 mL) to remove the Betadine. The cord is then sectioned into ˜5 cm pieces. When the cord piece has been completely dissected and cleaned of blood with PBS, it is placed into the 50 ml tube or 100 mm tissue culture plate containing 40 U/mL hyaluronidase/0.4 mg/mL collagenase solution for 30 minutes in a 37° C. humidified incubator with 5% CO₂. The digested piece of cord section is then placed into a sterilized cell strainer and pestle with a 40 mesh screen installed. The apparatus is then placed on a sterile 100 mm Petri dish, and 5-10 mL of Defined Media (DM) is added which contains: 58% low glucose DMEM (Invitrogen, Carlsbad, Calif.), 40% MCDB201 (Sigma, St. Louis, Mo.), 1× insulin-transferrin-selenium-A (Invitrogen, Carlsbad, Calif.), 0.15 g/mL AlbuMAX I (Invitrogen, Carlsbad, Calif.), 1 nM dexamethasone (Sigma, St. Louis, Mo.), 100 μM ascorbic acid 2-phosphate (Sigma, St. Louis, Mo.), 100 U penicillin, 1000 U streptomycin (Mediatech, Inc., Herdon, Va.), 2% fetal bovine serum (FBS) (Invitrogen, Carlsbad, Calif.), 10 ng/mL epidermal growth factor (EGF) (R & D Systems, Minneapolis, Minn.), and 10 ng/mL platelet-derived growth factor BB (PDGF-BB) (R & D Systems, Minneapolis, Minn.). The tissue is triturated and pushed through the strainer with a pestle until most of the tissue has lost its structure and the fluid is collected with a pipet. The sample is centrifuged at 750 RCF (×g) for 10 minutes. The media is aspirated off with care so as not to disturb the pellet. The pellet is resuspended in the appropriate volume of DM to obtain the desired range where antimicrobial control is obtained.

The diluted cell preparation is then seeded into 6-well plates or other vessels as appropriate. The cells are placed in a 37° C. humidified incubator with 5% CO₂ and left undisturbed for ˜24 hours. 24-48 hours after isolation, non-adherent cells are removed by washing three times with sterile PBS. Fresh DM is changed every two days. When culture confluency of between 50-80% is reached the cells are harvested using 0.05% trypsin/0.53 mM EDTA solution and re-plated into a T25 culture flask for further expansion in DM. Cultures are maintained at the stated confluency (50-80%) for propagation. Cultures are maintained in a 37° C. humidified incubator with 5% CO₂. Cultures are replenished with fresh DM every 2-3 days.

Once the stem cells have been isolated, the population is expanded mitotically. The stem cells should be transferred or “passaged” to fresh medium when they reach an appropriate density, such as 3×10⁴-cm² to 6.5×10⁴-cm², or, defined percentage of confluency on the surface of a culture dish. During incubation of the stem cells, cells can stick to the walls of the culture vessel where they can continue to proliferate and form a confluent monolayer. Alternatively, the liquid culture can be agitated, for example, on an orbital shaker, to prevent the cells from sticking to the vessel walls. The cells can also be grown on Teflon-coated culture bags.

In another embodiment, the desired mature cells or cell lines are produced using stem cells that have gone through a low number of passages, such as 2, 3, 4, 5, 6, 7, 8, 9, 10, 11, 12, 13, 14, or 15 passages. However, in some embodiments, cells are maintained for more doublings, such as 20, 25, 30, 35, 40, 45, 50, 55, 60, 65, 70, 75, 80, 90 or more than 100 population doublings. The invention contemplates that once stem cells have been established in culture, their ability to serve as progenitors for mature cells or cell lines can be maintained, for example, by regular passage to fresh medium as the cell culture reaches an appropriate density or percentage of confluency, or by treatment with an appropriate growth factors, or by modification of the culture medium or culture protocol, or by some combination of the above.

According to the invention, UCM cells may be obtained from Wharton's jelly collected from a subject's own umbilical cord. Alternatively, it may be advantageous to obtain UCM stem cells from Wharton's jelly obtained from an umbilical cord associated with a developing fetus or newly-born child, where the subject in need of treatment is one of the parents of the fetus or child. Alternatively, because of the “fetal” nature of cells isolated from Wharton's jelly, immune rejection of the cells of the invention and/or the new hepatocyte or hepatocyte-like cells produced therefrom may be minimized. As a result, such cells may be useful as “ubiquitous donor cells” for the production of new hepatocyte or hepatocyte-like cells for use in any subject in need thereof.

Differentiation of UCM Cells Into Hepatovctes

The UCM cells isolated as described herein are differentiated into cells of the hepatocyte lineage using the methods as described herein.

The term “hepatocyte-like” or “cell of the hepatocyte lineage” as used herein refer to cells that express at least two hepatocyte markers. Illustrative hepatocyte markers include, but are not limited to, expression of albumin, αFP, hepatocytes nuclear factor 4 alpha (HNF4α), hepatocytes nuclear factor 3 beta (HNF3-β), cytokeratin 18 (CK18), glutamine synthetase (GS), more disorganized smooth muscle actin (SMA), and Von Willebrand Factor (VWF). Illustrative markers also include hepatocyte-inducible genes such as androstane receptor (CAR), pregnane X receptor (PXR), peroxisome proliferators-activated receptor γ coactivator-1α (PGC-1), Phosphoenolpyruvate carboxykinase (PEPCK) and peroxisome proliferators-activated receptor-γ (PPAR-γ), (key gluconeogenic enzymes), CYP3A4 (a cytochrome P450 (CYP) Phase I monooxygenase system enzyme important for endo- and xenobiotic metabolism). In certain embodiments, these inducible genes have either elevated expression in the differentiated hepatocyte-like cells or can be induced in upon treatment with PB, RIF, 8-Br-cAMP or forskolin. Additional relevant hepatocyte markers that may be expressed by the hepatocyte-like cells of the invention include albumin production; product of 7-pentoxyresorufin-O-dealkylation (PROD), which is catalyzed specifically by CYP2B1/2; the enzyme required for hepatic bilirubin elimination, UDP-glucuronosyltransferase (UGT1A1); Human hydroxysteroid sulfotransferase (SULT2A1) which catalyzes the sulfonation and detoxication of endogenous and xenobiotic substrates; transthyretin (TTR), tryptophan-2,3-dioxygenase (TDO); alfa-1-antitrypsin (alfa-1-AT), Liver-Specific Organic Anion Transporter (LST-1, also called OATP2); and carbamoyl phosphate synthase 1 (CPSase-1). Further illustrative markers include morphological characteristics such as being mostly mononuclear and heterogeneous with high nucleus to cytoplasmic ratio, more polygonal to cuboidal shape, displaying lipid droplet inclusions, ability to form cannicular type structures, and ability to develop sinusoids. Yet further illustrative markers include characteristics such as glycogen production, synthesis of serum proteins, plasma proteins, clotting factors, detoxification functions, urea production, gluconeogenesis and lipid metabolism. Thus, in certain embodiments, the hepatocyte-like cells express more mature hepatocyte functions, such as functioning metabolic pathways.

In certain embodiments, the hepatocyte-like cells of the invention express three or more hepatocyte markers as described herein. In another embodiment, the hepatocyte-like cells express four or more of the hepatocyte markers as described herein. In certain embodiments, the hepatocyte-like cells of the invention express five or more hepatocyte markers as described herein. In other embodiments, the hepatocyte-like cells of the invention express six, seven, eight, nine, ten or more hepatocyte markers as described herein. As would be appreciated by the skilled artisan, the hepatocyte-like cells of the invention may also express other known markers or functions.

In one embodiment, the UCM are differentiated using the following method: Prior to induction, the UCM are cultured in Defined Media containing: Low glucose DMEM, MCDB201, 1×ITS, 0.15 g/mL Albumax, 1 nM Dexamethasone, 100 uM Ascobic acid-2-Phosphate, 10 ng/mL EGF, 10 ng/mL PDGF, 2% FBS, Pen/Strep. UCM are then cultured for 2 days in Pre-induction Media containing: Serum Free Iscove's Modified Dulbecco's Medium (IMDM), 20 ng/ml EGF, 10 ng/ml bFGF, Pen/Strep. The cells are then cultured for 7 days in Differentiation Media containing IMDM, 20 ng/ml HGF, 10 ng/ml bFGF, 0.61 g/L nicotinamide, 2% FBS, Pen/Strep. The cells are then cultured to 10 weeks in Maturation Media containing IMDM, 20 ng/ml oncostatin M, 1 umol/L dexamethasone, 50 mg/ml ITS+premix, 2% FBS, Pen/Strep.

In another embodiment, the differentiation protocol is a sequential addition of exogenous factors. Prior to induction, cells are seeded on 0.1% gelatin coated T75 culture flasks at a density of 2.0-3.0E06 cells/flask and allowed to adhere overnight. Cells are then treated for two days in pre-induction media comprising Serum free IMDM (Invitrogen, Carlsbad, Calif.), 20 ng/ml recombinant human epidermal growth factor (rhEGF) (R & D Systems, Minneapolis, Minn.), 10 ng/ml recombinant human basic fibriblast growth factor (rhbFGF) (Chemicon, Temecula, Calif.), and Pen/Strep. Differentiation is accomplished using a two step process where cells are culture for 7 days in IMDM, 20 ng/ml recombinant human hepatocytes growth factor (rhHGF) (Chemicon, Temecula, Calif.), 10 ng/ml rhbFGF, 0.61 g/L nicotinamide (Sigma, St. Louis, Mo.), 2% FBS, Pen/Strep. Cells are then cultured up to 10 weeks in maturation media containing: IMDM, 20 ng/ml Human Oncostatin M (Bioscource, Camarillo, Calif.), 1 umol/L dexamethasone, 50 mg/ml ITS+ premix (Sigma, St. Louis, Mo.), 2% FBS, and Pen/Strep. Media is changed every three days and hepatic differentiation is assessed in a temporal manner.

In further embodiments, the UCM cells are differentiated by first culturing in the standard culturing medium used for UCM cells as described herein, such as, Defined Media comprising: Low glucose DMEM, MCDB201, 1×ITS, 0.06, 0.07, 0.08, 0.09, 0.10, 0.15, 0.16, 0.17, 0.18, 0.19, 0.2, 0.3, 0.4, 0.5 g/mL or higher Albumax; 0.1, 0.2, 0.3, 0.4, 0.5, 0.6, 0.7, 0.8, 0.9, or 1 nM Dexamethasone or higher concentrations such as 1.1, 1.2, 1.3, 1.4, 1.5, 1.6, 1.7, 1.8, 1.9, 2.0, 2.5, 3.0 or 3.5 nM dexamethasone; 50, 60, 70, 80, 90, 100, 110, 120, 130, 140, or 150 uM Ascobic acid-2-Phosphate; 1, 2, 3, 4, 5, 6, 7, 8, 9, 10, 11, 12, 13, 14, 15, 16, 17, 18, 19, or 20 ng/mL EGF; 1, 2, 3, 4, 5, 6, 7, 8, 9, 10, 11, 12, 13, 14, 15, 16, 17, 18, 19, or 20 ng/mL PDGF; 0.5, 1.0, 1.5, 2, 2.5, 3, 3.5, 4, 4.5 or 5% FBS; and Pen/Strep. UCM cells are then cultured for 1, 2, 3, 4, or 5 days or longer in Pre-Induction Media comprising: Serum Free Iscove's Modified Dulbecco's Medium (IMDM); 10, 11, 12, 13, 14, 15, 16, 17, 18, 19, 20, 21, 22, 23, 24, 25, 26, 27, 28, 29, or 30 ng/ml, or higher concentrations, of EGF; 1, 2, 3, 4, 5, 6, 7, 8, 9, 10, 11, 12, 13, 14, 15, 16, 17, 18, 19, or 20 ng/ml bFGF; and Pen/Strep. The cells are then cultured for 2, 3, 4, 5, 6, 7, 8, 9, 10, 11, 12, 13, 14, or more days in Differentiation Media comprising IMDM; 10, 11, 12, 13, 14, 15, 16, 17, 18, 19, 20, 21, 22, 23, 24, 25, 26, 27, 28, 29, or 30 ng/ml HGF; 1, 2, 3, 4, 5, 6, 7, 8, 9, 10, 11, 12, 13, 14, 15, 16, 17, 18, 19, or 20 ng/ml bFGF; 0.1, 0.2, 0.3, 0.4, 0.5, 0.61, 0.7, 0.8, 0.9 g/L, or more, nicotinamide; 0.5, 1.0, 1.5, 2, 2.5, 3, 3.5, 4, 4.5 or 5% FBS; and Pen/Strep. The cells are then cultured to 3, 4, 5, 6, 7, 8, 9, 10, 11, 12, 13, 14, 15, 16, 17, 18, 19, or 20 weeks or longer in Maturation Media comprising IMDM; 10, 11, 12, 13, 14, 15, 16, 17, 18, 19, 20, 21, 22, 23, 24, 25, 26, 27, 28, 29, or 30 ng/ml oncostatin M; 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2.0, 3.0, 4.0 or 5 umol/L, or higher, dexamethasone; 10, 20, 30, 40, 50, 60, 70, 80, 90, or 100 mg/ml ITS+ premix (BD Biosciences) or more; 0.5, 1.0, 1.5, 2, 2.5, 3, 3.5, 4, 4.5 or 5% FBS; and Pen/Strep.

In certain embodiments, the cells are differentiated in the presence of a variety of growth factors, including but not limited to, hepatocyte growth factor (HGF), epidermal growth factor (EGF), transforming growth factor (TGF), acid fibroblast growth factor (aFGF), insulin, insuline-like growth factor (IGF), granulocyte macrophage colony-stimulating factor (GM-CSF), stromal derived factor-1α (SDF-1α), stem cell factor (SCF), oncostantin M (OSM), serum-derived hepatocyte growth stimulating factor (HGSF), dexamethasone, retinoic acid, sodium butyrate, nicotinamide, norepinephrine, and dimethyl sulfoxide. In one embodiment, the growth factors are recombinant human growth factors.

In one embodiment, hepatocyte-like cells are differentiated in the presence of a scaffold to allow three-dimensional culturing of the cells during differentiation. The scaffold material may comprise naturally occurring components or may be comprised of synthetic materials, or both. The scaffold material may be biocompatible. Illustrative scaffold material includes extracellular matrices, and materials described in, for example, Hamamoto R, et al. J Biochem (Tokyo) 1998; 124(5):972-979; HENG BC, et al., Journal of Gastroenterology and Hepatology. 2005; 20(7):975-987. Other scaffold materials that can be used in the context of the present invention include but are not limited to one or a mixture of two or more of the following: collagens (e.g., collagen types I, III, IV, V and VI), gelatin, alginate, fibronectin, laminin, entactin/nidogen, tenascin, thrombospondin, SPARC, undulin, proteoglycans, glycosaminoglycans (e.g., hyaluronan, heparan sulfate, chondroitin sulfate, keratan sulfate and dermatan sulfate), polypropylene, TER polymer, alginate-poly L-lysine, chondroitin sulfate, chitosan, MATRIGEL® (Becton-Dickinson, Inc USA) or other commercially available extracellular matrix materials. In one particular embodiment, the extracellular matrix for use in differentiating the UCM into hepatocyte-like cells is gelatin.

In one embodiment, the UCM cells are differentiated by coculture with a hepatocyte feeder layer, such as with isolated liver cells, immortalized hepatocytes such as those described in U.S. Pat. Nos. 5,869,243 and 6,107,043, or with other hepatocyte cell lines available in the art, e.g., HB8065 cells. In this regard, the UCM cells may be cultured in a standard growth medium, such as DMEM supplemented with 2% FBS, and cultured with a heat-shocked or otherwise disabled hepatocyte feeder layer. Such culture may be carried out on a porous membrane in a transwell insert.

In certain embodiments, the UCM cells are cultured in one or more of the media described herein, such as, Defined Media, Pre-induction Media, Differentiation Media, and Maturation Media for a time sufficient for the UCM cells to differentiate into cells of the hepatocyte lineage, as indicated by any of a number of indicators, including morphological changes, expression of hepatocyte genes, expression of hepatocyte proteins, and hepatocyte functional characteristics, as described further herein.

Thus, in certain embodiments, the UCM cells are cultured in one or more of the media described herein, such as, Defined Media, Pre-induction Media, Differentiation Media, and Maturation Media for a time sufficient for the UCM cells to express albumin at levels above cells cultured in control media. In a further embodiment, the UCM cells are cultured in one or more of the media described herein, such as Defined Media, Pre-induction Media, Differentiation Media, and Maturation Media for a time sufficient for the UCM cells to express α-Fetal Protein (αFP) above levels of cells cultured in control media. Generally, undifferentiated UCM control cells do not express albumin or αFP. In a further embodiment, the UCM cells are cultured in one or more of the media described herein for a time sufficient for the smooth muscle actin to become less organized than in undifferentiated cells. In a further embodiment, the UCM cells are cultured in one or more of the media described herein for a time sufficient for the cells to adopt a hepatocyte-like morphology, including but not limited to, a flattened polygonal shape as compared to the spindle-shaped morphology of the undifferentiated cells. In one embodiment, the UCM cells are cultured in one or more of the media described herein for a time sufficient for one or more of the following: the cells to express albumin, to express α-FP, adopt a hepatocyte-like morphology and for the smooth muscle actin to become less organized.

In one embodiment, the UCM cells are cultured in one or more of the media described herein for a time sufficient for expression of at least two of the following markers: albumin, αFP, hepatocytes nuclear factor 4 alpha (HNF4α), cytokeratin 18 (CK18), glutamine synthetase (GS), more disorganized smooth muscle actin (SMA), Von Willebrand Factor (VWF), a hepatocyte-inducible gene such as androstane receptor (CAR), pregnane X receptor (PXR), peroxisome proliferators-activated receptor γ coactivator-1α (PGC-1), Phosphoenolpyruvate carboxykinase (PEPCK) and peroxisome proliferators-activated receptor-γ (PPAR-γ), (key gluconeogenic enzymes), CYP3A4 (a cytochrome P450 (CYP) Phase I monooxygenase system enzyme important for endo- and xenobiotic metabolism) (These inducible genes have either elevated expression in the differentiated hepatocyte-like cells or can be induced in upon treatment with PB, RIF, 8-Br-cAMP or forskolin); morphological characteristics such as being mostly mononuclear and heterogeneous with high nucleus to cytoplasmic ratio, more polygonal to cuboidal shape, displaying lipid droplet inclusions, ability to form cannicular type structures, ability to develop sinusoids, glycogen production, synthesis of serum proteins, plasma proteins, clotting factors, detoxification functions, urea production, gluconeogenesis and lipid metabolism.

In one particular embodiment, the UCM cells are differentiated into hepatocyte-like cells by culturing in IMDM with gelatin, recombinant human growth factors (e.g., rhEGF, rhbFGF, rhHGF, Human Oncostatin M), and KNOCKOUT™ Serum Replacement (Invitrogen, Carlsbad, Calif.).

In a further embodiment, the cells are cultured for a sufficient time to acquire hepatocyte-like functional properties, such as glycogen production, synthesis of serum proteins, plasma proteins, clotting factors, detoxification functions, urea production, gluconeogenesis and lipid metabolism. In this regard, differentiation is assessed by measuring functional properties such as glycogen production, using techniques known in the art. Glycogen is a simple intracytoplasmic polysaccharide found in abundance in the liver cells. To demonstrate glycogen storage, differentiated cells may be stained with Periodic Acid-Schiff (PAS). Glycogen can be digested by diastase in cell culture conditions. To demonstrate positive glycogen staining differentiated cells may be pretreated with Diastase solution.

Cellular uptake of anionic dye, Indocyanine Green (ICG), can be examined in differentiated cells to determine hepatic function. This can be carried out using techniques known in the art. In one embodiment, ICG is dissolved to an initial concentration of 5 mg/mL in solvent. The solution is then diluted to 1 mg/mL in maturation media and added to the culture dish and incubated at 37° C. in a humidified incubator at 5% CO₂ for 10-15 minutes. The cells are washed thoroughly with sterile PBS and then visualized under a light microscope. After examination, the PBS was then removed and maturation media is added and the cells incubated at 37° C. in a humidified incubator at 5% CO₂ for ˜4-6 hours to confirm elimination of ICG.

Liver cells express LDL receptors for regulation of cholesterol homeostasis in mammals. Thus, uptake of LDL can be used as an indicator of differentiation. To determine if differentiated cells exhibited cellular uptake of LDL, cells are treated with Dil-Ac-LDL. In one embodiment, Dil-Ac-LDL is diluted in maturation media to 10 μg/mL, added to cells, and incubated for 4 hours at 37° C. in a humidified incubator. After incubation, media is removed containing the Dil-Ac-LDL and the cells were washed 2× with probe-free maturation media. Cells may be visualized using standard rhodamine excitation:

As would be recognized by the skilled artisan upon reading the present disclosure, any of a variety of techniques known in the art can be used to determine expression of albumin, α-FP, organization of smooth muscle actin and cell morphology, including but not limited to gene expression assays such as PCR, RT-PCR, quantitative PCR, protein expression analyses including immunohisochemistry, immunofluorescence assays, and the like. Such techniques are known in the art and are described for example, in Current Protocols in Molecular Biology, or Current Protocols in Cell Biology, both John Wiley and Sons, NY, N.Y.

Differentiation of the cells of the invention can be detected by a variety of techniques, such as, but not limited to, flow cytometric methods, immunohistochemistry, immunofluorescence techniques, in situ hybridization, and/or histologic or cellular biologic techniques.

The invention includes a method of generating a bank of hepatocyte-like cells that have been differentiated from UCM stem cells, by obtaining matrix cells from umbilical cord, fractionating the matrix into a fraction enriched with a stem cell and culturing the stem cells in a culture medium containing one or more growth factors so as to differentiate the cells into hepatocyte-like cells, as described herein. Alternatively, a bank of the umbilical cord itself and/or unfractionated cells may be maintained for obtaining matrix cells at a later date.

The invention also contemplates the establishment and maintenance of cultures of hepatocyte-like cells differentiated from UCM.

Once the cells of the invention have been established in culture, as described above, they may be maintained or stored in “cell banks” comprising either continuous in vitro cultures of cells requiring regular transfer, or, in certain embodiments, cells which may be cryopreserved. Hepatocyte-like cells differentiated from UCM stem cells derived from umbilical cords obtained from genetically diverse populations are obtained and stored in the banks to be used at a future time.

Cryopreservation of cells of the invention may be carried out according to known methods, such as those described in Doyle et al., 1995, Cell and Tissue Culture. For example, but not by way of limitation, cells may be suspended in a “freeze medium” such as, for example, culture medium further comprising 15-20% FBS and 10% dimethylsulfoxide (DMSO), with or without 5-10% glycerol, at a density, for example, of about 4−10×10⁶ cells/ml. The cells are dispensed into glass or plastic ampoules (Nunc) that are then sealed and transferred to the freezing chamber of a programmable freezer. The optimal rate of freezing may be determined empirically. For example, a freezing program that gives a change in temperature of about −1° C./min through the heat of fusion may be used. Once the ampoules have reached about −180° C., they are transferred to a liquid nitrogen storage area. Cryopreserved cells can be stored for a period of years, though they should be checked at least every 5 years for maintenance of viability.

The cryopreserved cells of the invention constitute a bank of cells, portions of which can be “withdrawn” by thawing and then used to produce new hepatocyte-like cells, etc. as needed, or to be used in any of the methods of use as described herein. Thawing should generally be carried out rapidly, for example, by transferring an ampoule from liquid nitrogen to a 37° C. water bath. The thawed contents of the ampoule should be immediately transferred under sterile conditions to a culture vessel containing an appropriate medium such as RPMI 1640, DMEM conditioned with 20% FBS. The cells in the culture medium are preferably adjusted to an initial density of about 3×10⁵ to 6×10⁵ cells/ml so that the cells can condition the medium as soon as possible, thereby preventing a protracted lag phase. Once in culture, the cells may be examined daily, for example, with an inverted microscope to detect cell proliferation, and sub-cultured as soon as they reach an appropriate density.

The cells of the invention may be withdrawn from the bank as needed, and used for drug screening or in the treatment of liver disorders as discussed further herein. The cells of the invention may be used either in vitro, or in vivo, for example, by direct administration of cells to a damaged liver where new cells are needed. As described supra, the hepatocyte-like cells of the invention may be used to produce new hepatocyte-like cells for use in a subject where the cells were originally isolated from that subject's umbilical cord (autologous). Alternatively, the cells of the invention may be used as ubiquitous donor cells, i.e., to produce new liver cells for use in any subject (heterologous).

The differentiated hepatocyte-like cells of the invention may also be provided as a panel of hepatocyte-like cells derived from multiple different umbilical cord sources from individuals of diverse genetic backgrounds and even from different animal sources. For example, the panel of UMC-derived hepatocyte-like cells may include hepatocyte-like cells derived from UMC sources from individuals known to have polymorphisms in genes encoding drug-metabolizing enzymes and drug transporters. The panels of the invention may be provided as part of a drug screening kit including reagents for drug screening, such reagents including, for example, any of the culture media described herein, and reagents for detecting albumin and α-FP expression.

In one embodiment, the hepatocyte-like cells of the invention can be genetically modified. In accordance with this embodiment, the hepatocyte-like cells of the invention are exposed to a gene transfer vector comprising a nucleic acid including a transgene, such that the nucleic acid is introduced into the cell under conditions appropriate for the transgene to be expressed within the cell. The transgene generally is an expression cassette, including a coding polynucleotide operably linked to a suitable promoter. The coding polynucleotide can encode a protein, or it can encode biologically active RNA, such as antisene RNA, siRNA or a ribozyme. Thus, the coding polynucleotide can encode a gene conferring, for example, resistance to a toxin or an infectious agent, such as Hepatitis A, B, or C, a hormone (such as peptide growth hormones, hormone releasing factor, sex hormones, adrenocorticotrophic hormones, cytokines such as interferons, interleukins, and lymphokines), a cell surface-bound intracellular signaling moiety such as cell-adhesion molecules and hormone receptors, and factors promoting a given lineage of differentiation, or any other transgene with known sequence.

Other illustrative transgenes for use herein encode growth effector molecules. Growth effector molecules, as used herein, refer to molecules that bind to cell surface receptors and regulate the growth, replication or differentiation of target cells or tissue, in particular liver cells. Illustrative growth effector molecules are growth factors and extracellular matrix molecules. Examples of growth factors include epidermal growth factor (EGF), platelet-derived growth factor (PDGF), transforming growth factors (TGFα, TGFβ), hepatocyte growth factor, heparin binding factor, insulin-like growth factor I or II, fibroblast growth factor, erythropoietin, nerve growth factor, and other factors known to those of skill in the art. Additional growth factors are described in “Peptide Growth Factors and Their Receptors I” M. B. Sporn and A. B. Roberts, eds. (Springer-Verlag, New York, 1990).

The expression cassette containing the transgene should be incorporated into the genetic vector suitable for delivering the transgene to the cell. Depending on the desired end application, any such vector can be so employed to genetically modify the cells (e.g., plasmids, naked DNA, viruses such as adenovirus, adeno-associated virus, herpesvirus, lentivirus, papillomavirus, retroviruses, etc.). Any method of constructing the desired expression cassette within such vectors can be employed, many of which are well known in the art, such as by direct cloning, homologous recombination, etc. The desired vector will largely determine the method used to introduce the vector into the cells, which are generally known in the art. Suitable techniques include protoplast fusion, calcium-phosphate precipitation, gene gun, electroporation, and infection with viral vectors.

Thus, the invention encompasses expression vectors and methods for the introduction of exogenous DNA into the cells with concomitant expression of the exogenous DNA in the cells such as those described, for example, in Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York).

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a nucleic acid encodes a protein if transcription and translation of mRNA corresponding to that nucleic acid produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

Methods of Use

The hepatocyte-like cells differentiated from UCM cells of the invention are useful in a variety of settings, including drug screening, screening for drug interactions, transplantation, tissue/organ regeneration and treatment of liver damage or other liver disorders.

In one embodiment, the invention provides methods for testing the activity of a compound (e.g., a drug or candidate drug). The activity of a compound may be assessed by measuring the effect of the drug on the viability, metabolic activity, the effect on P450 enzyme gene expression or protein activity of the hepatocyte-like cells of the invention or the effect of the drug on drug transport transporters. As would be understood by the skilled artisan, the hepatocyte-like cells of the invention may be used in any known drug screening assay, such as assays on specific P450 enzymes or panels of P450 enzymes, current drug screening assays that use hepatocyte cells, and the like. The present invention provides the advantage that the hepatocyte-like cells of the invention are easily procured and can be derived from individuals with diverse genetic backgrounds.

In one embodiment, the present invention provides methods for testing the activity (such as the toxicity) of a compound by contacting the hepatocyte-like cells of the invention with a compound and measuring the viability of the hepatocyte-like cells. A decrease in viability in the presence of a test compound compared to that in the absence of the test compound indicates that the compound is toxic in vivo. Viability of cells can be determined using techniques well known to the skilled artisan, such as staining followed by flow cytometry or simply by visualizing the cells with a microscope using a hemacytometer.

In another embodiment, the present invention provides methods for testing the activity of a compound by contacting the hepatocyte-like cells of the invention with a compound and measuring the metabolic activity of the hepatocyte-like cells. A decrease or increase in metabolic activity in the presence of a test compound compared to that in the absence of the test compound indicates a drug activity in vivo.

In another embodiment, the present invention provides methods for testing the activity of a compound by contacting a first hepatocyte-like cell of the invention with the compound to produce a cell supernatant and then contacting a second hepatocyte-like cell with the cell supernatant and measuring viability and/or the metabolic activity of the second hepatocyte-like cell. A decrease in viability and/or a decrease or increase in metabolic activity of the second hepatocyte-like cell in the presence of the supernatant compared to that in the absence of the cell supernatant indicates that the compound may have activity in vivo. For example, a decrease in viability of the second hepatocyte-like cell in the presence of the supernatant compared to that in the absence of the cell supernatant indicates that the compound is toxic in vivo.

One embodiment of the present invention provides methods for testing the activity of a compound by contacting the hepatocyte-like cells of the invention with a compound and measuring the induction or inhibition of one or more cytochrome P450 enzyme gene expression or protein activity. An increase or decrease in one or more cytochrome P450 gene expression and/or enzyme activity in the presence of a test compound compared to that in the absence of the test compound provides important activity information about the compound in vivo particularly with regard to potential drug interactions with known drugs.

In yet a further embodiment, the present invention provides methods for testing the activity of a compound by contacting a first hepatocyte-like cell of the invention with the compound to produce a cell supernatant and then contacting a second hepatocyte-like cell with the cell supernatant and measuring the induction of one or more cytochrome P450 enzyme gene expression or protein activity in the second hepatocyte-like cell. An increase or decrease in gene expression and/or enzyme activity of the second hepatocyte-like cell in the presence of the supernatant compared to that in the absence of the cell supernatant indicates the particular activity of the compound in vivo. This activity information is important for example, with regard to known drugs and can also be used for drug interaction testing for future drugs.

A further embodiment of the invention provides methods for evaluating drug interactions. Drug interactions can be evaluated by contacting the cells of the invention with two compounds and determining whether the effect on the cells of one compound is impacted by the presence of the second compound. For example, the method may comprise contacting a first population of the hepatocyte-like cells with a first compound, contacting a second population of the hepatocyte-like cells with a second compound and contacting a third population of hepatocyte-like cells with both the first and the second compounds and measuring a particular effect in each of the populations (e.g., cell viability, metabolic activity, a cytochrome P450 gene/protein expression or activity) wherein a statistically significant decrease or increase in an effect in the third population contacted with both compounds as compared to either of the first or second populations would indicate a drug interaction. A drug interaction may comprise one drug inhibiting another drug or one drug increasing the activity of another drug.

As noted above, cytochrome P450 profiles on known drugs are available in the art. As such, drug interactions can be determined for a candidate compound by evaluating its effect on cytochrome P450 enzymes using the hepatocyte-like cells using the methods as described herein and comparing the results to the known profiles of known drugs, providing valuable information with regard to interactions of a candidate compound with known drugs (e.g., commonly used over-the-counter drugs such as ibuprofen, acetaminophen, aspirin, and the like).

As would be recognized by the skilled artisan, gene expression can be measured using any of a variety of techniques known in the art, such as but not limited to, quantitative polymerase chain reaction (QC-PCR or QC-RT PCR). Other methods for detecting mRNA expression are well-known and established in the art and may include, but are not limited to, transcription-mediated amplification (TMA), polymerase chain reaction amplification (PCR), reverse-transcription polymerase chain reaction amplification (RT-PCR), ligase chain reaction amplification (LCR), strand displacement amplification (SDA), and nucleic acid sequence based amplification (NASBA).

Enzyme activity can be measured using assays known in the art, such as but not limited to, enzyme assays of hepatocyte microsome preparations (see e.g., R. Walsky, and R. Scott Obach Drug Metabolism and Disposition 32: 647-660, 2004). Other assays are commercially available such as, High Throughput P450 Inhibition Kits, BD Biosciences (San Jose, Calif.); or other kits available through Invitrogen (Carlsbad, Calif.), Promega (Madison, Wis.), Sigma Aldrich (St. Louis, Mo.), and other companies. Human liver microsomes provide a convenient way to study CYP450 metabolism. Microsomes are a subcellular fraction of tissue obtained by differential high-speed centrifugation. All of the CYP450 enzymes are collected in the microsomal fraction. The CYP450 enzymes retain their activity for many years in microsomes or whole liver stored at low temperature (e.g., −70° C.). Cofactor requirements for o CYP450-mediated reactions are well characterized, consisting primarily of a redox sustaining system such as NADPH. Hepatic microsomes can be obtained using techniques known in the art (see e.g., Coughtrie et al., Clin Chem 1991 37/5 739-742; J. Lam and L. Benet Drug Metabolism and Disposition 32: 1311-1316, 2004; Salphati L and Benet L Z (1999) Metabolism of digoxin and digoxigenin digitoxosides in rat liver microsomes: involvement of cytochrome P4503A. Xenobiotica 29: 171-185) The cDNAs for the common CYP450s have been cloned, and the recombinant human enzymatic proteins have been expressed in a variety of cells. After the apparent metabolic pathway has been determined using microsomes, use of these recombinant enzymes provides an excellent way to confirm results.

Suitable metabolic enzymes that can be measured in a drug screening assay using the hepatocyte-like cells of the invention include but are not limited to cytochrome P450 enzymes. Suitable CYP 450 enzymes include CYTOCHROME P450, CYPlAl, CYP1A2, CYP2A1, 2A2, 2A3, 2A4, 2A5, 2A6, CYP2B1, 2B2, 2B3, 2B4, 2B5, 2B6, CYP2C1, 2C2, 2C3, 2C4, 2C5, 2C6, 2C7, 2C8, 2C9, 2C10, 2C11, 2C12, CYP2D1, 2D2, 2D3, 2D4, 2D5, 2D6, CYP2E1, CYP3A1, 3A2, 3A3, 3A4, 3A5, 3A7, CYP4A1, 4A2, 4A3, 4A4, CYP4A11, CYP P450 (TXAS), CYP P450 11A (P450scc), CYP P450 17(P45017a), CYP P450 19 (P450arom), CYP P450 51 (P45014a), CYP P450 105A1, CYP P450 105B1. Generally a drug screening assay using the hepatocyte-like cells of the present invention include measuring for cytochrome P450 enzyme induction. In this regard, induction can be measured at the gene expression level or can be measured by the protein activity of the specific enzymes (see e.g., U.S. Pat. Nos. 6,830,897; 7,041,501). Commercially available tests may be applicable for use with the hepatocyte-like cells of the invention. These include, but are not limited to, TranscriptionPath (GenPathway, Inc. San Diego, Calif.); HTS P450 Inhibition Kits, BD Biosciences, San Jose, Calif.); and the like.

Other important metabolic enzymes that can be measured in a drug screening assay using the hepatocyte-like cells of this invention including enzymes responsible for acetylation, methylation, glucuronidation, sulfation, and de-esterification (esterases). Suitable metabolic enzymes whose activity (including enzyme activity or gene expression) can be measured include glutathione-thioethers, Leukotriene C4,butyrylcholinesterase, N-Acetyltransferase, UDP-glucuronosyltransferase (UDPGT) isoenzymes, TL PST, TS PST, drug glucosidation conjugation enzyme, the glutathione-S-transferases (GSTs) (RX:glutathione-R-transferase), GST1, GST2, GST3, GST4, GST5, GST6, alcohol dehydrogenase (ADH), ADH I, ADH II, ADH III, aldehyde dehydrogenase (ALDH), cytosolic (ALDH1), mitochondrial (ALDH2), monoamine oxidase, MAO: Ec 1.4.3.4, MAOA, MAOB, flavin-containing monoamine oxidase, enzyme superoxide dismutase (SOD), Catalase, amidases, N1,-monoglutathionyl spermidine, N1,N8-bis(glutathionyl) spermidine, Thioesters, GS-SG, GS-S-cysteine, GS-S-cysteinylglycine, GS-S-O3H, GS-S-CoA, GS-S-proteins, S-carbonic anhydrase III, S-actin, Mercaptides, GS-Cu(I), GS-Cu(II)-SG, GS-SeH, GS-Se-SG, GS-Zn-R, GS-Cr-R, Cholin esterase, lysosomal carboxypeptidase, Calpains, Retinol dehydrogenase, Retinyl reductase, acyl-CoA retinol acyltrunderase, folate hydrolases, protein phosphates (pp) 4 st, PP-1, PP-2A, PP-2 Bpp-2C, deamidase, carboxyesterase, Endopeptidases, Enterokinase, Neutral endopeptidase E.C.3.4.24.11, Neutral endopeptidase, carboxypeptidases, dipeptidyl carboxypeptidase, also called peptidyl-dipeptidase A or angiotensin-converting enzyme (ACE) E.C.3.4.15.1, carboxypeptidase M, g-Glutamyl transpeptidase E.C.2.3.2.2, Carboxypeptidase P, Folate conjugase E.C.3.4.12.10, Dipeptidases, Glutathione dipeptidase, Membrane Gly-Leu peptidases, Zinc-stable Asp-leu dipeptidase, Enterocytic intracellular peptidases, Amino tripeptidase E.C.3.4.11.4, Amino dipeptidase E.C.3.4.13.2, Prodipeptidase, Arg-selective endoproteinase; the family of brush border hydrolases, Endopeptidase-24.11, Endopeptidase-2(meprin), Dipeptidyl peptidase IV, Membrane dipeptidase GPI, Glycosidases, Sucrase-isomaltase, Lactase-glycosyl-ceraminidase, Glucoamylase-maltase, Trehalase, Carbohydrase enzymes, alfa-Amylase (pancreatic), Disaccharidases (general), Lactase-phhlorizin hydrolase, Mammalian carbohydrases, Glucoamylase, Sucrase-Isomaltase, Lactase-glycosyl ceramidase, Enzymatic sources of ROM, Xanthine oxidase, NADPH oxidase, Amine oxidases, Aldehyde oxidase, Dihydroorotate dehydrogenase, Peroxidases, Trypsinogen 1, Trypsinogen 2, Trypsinogen 3, Chymotrypsinogen, proElastase 1, proElastase 2, Protcase E,Kallikreinogen, procarboxypeptidase A1, procarboxypeptidase A2, procarboxypeptidase B1, procarboxypeptidase B2, Glycosidase, Amylase, lipases, Triglycaride lipase, Collipase, Carboxyl ester hydrolase, Phospholipase A2, Nucleases, Dnase I, Ribonucleotide reductase (RNRs), Label Protein IEP, A1 Amylase 1, A2 Amylase 2, Lipase, CEL Carboxyl-ester lipase, PL Prophospholipase A, T1 Trypsinogen 1, T2 Trypsinogen 2, T3 Trypsinogen 3, T4 Trypsinogen 4, C₁-Chymotrypsinogen 1, C2 Chymotrypsinogen 2, PE1 Proelastase 1, PE2 Proelastase 2, PCA Procarboxypeptidase A1, PCA1 Procarboxypeptidase A2, PCB1 Procarboxypeptidase B1, PCB2 Procarboxypeptidase B2, R Ribonuclease, LS Lithostatin, Characteristics of UDPGT isoenzymes purified from rat liver, 4-nitrophenol UDPGT, 17b-Hydroxysteriod UDDPGT, 3-a-Hydroxysteroid UDPGT, Morphine UDPGT, Billirubin UDPGT, Billirubin monoglucuronide, Phenol UDPGT, 5-Hydroxytryptamine UDPGT, Digitoxigenin monodigitoxide UDPGT, 4-Hydroxybiphenyl UDPGT, Oestrone UDPGT, Peptidases, Aminopeptidase N, Aminopeptidase A, Aminopeptidase P, Dipeptidyl peptidase IV, b-Casomorphin, Angiotensin-converting enzyme, Carboxypeptidase P Angiotensin II, Endopeptidase-24.11, Endopeptidase-24.18 Angiotensin I, Substance P (deamidated), Exopeptidase,1. NH₂ terminus Aminopeptidase N (EC 3.4.11.2), Aminopeptidase A (EC 3.4.11.7), Aminopeptidase P (EC 3.4.11.9), Aminopeptidase W (EC 3.4.11.-), Dipeptidyl peptidase IV (EC 3.4.14.5), g-Glutamyl transpeptidase (EC 2.3.2.2), 2. COOH terminus Anglotensin-converting enzyme (EC 3.4.15.1), Carboxypeptidase P (EC 3.4.17.-), Carboxypeptidase M (EC 3.4.17.12),3. Dipeptidase Microsomal dipeptidase (EC 3.4.13.19), Gly-Leu peptidase, Zinc stable peptidase,Endopeptidase Endopeptidase-24.11 (EC 3.4.24.11), Endopeptidase-2 (EC 3.4.24.18, PABA-peptide hydrolase, Meprin, Endopeptidase-3, Endopeptidase (EC 3.4.21.9), GST A1-1, Alpha,GST A2-2 Alpha, GST M1a-1a Mu, GST M1b-1b Mu, GST M2-2 Mu, GST M3-3 Mu, GST M4-4 Mu, GST M5-5 Mu, GST P1-1 Pi, GST T1-1 Theta, GST T2-2 Theta, Microsomal Leukotriene C4 synthase, UGT isozymes, UGT1.1, UGT1.6, UGT1.7, UGT2.4, UGT2.7, UGT2.11, Elastase, Aminopeptidase (dipeptidyl aminopeptidase (IV), Chymotrypsin, Trypsin, Carboxypeptidase A, Methyltransferases, O-methyltransferases, N-methyltransferases, S-methyltransferases, Catechol-O-methyltransferases, MN-methyltransferase, S-sulphotransferases, Mg²⁺-ATPase, Growth factor receptors Alkaline phosphatase, ATPases, Na, K⁺ ATPase, Ca²⁺-ATPase, Leucine aminopeptidase, K⁺ channel.

Measuring metabolic activity is carried out using techniques known in the art, such as, for example, by contacting the cells with a test compound and collecting supernatant. Metabolites of the compound present in the supernatant are measured using known techniques, such as through an appropriate type of high performance liquid chromatography (HPLC). Thymidine incorporation by cultured hepatocyte-like cells can be measured to assess cell proliferation in vitro. See also, Handbook of Drug Metabolism Ed. Thomas Woolf, Informa Healthcare; Mar. 29, 1999.

Media from cell cultures, i.e., culture supernatants is generally collected and stored at −30° C. until assayed. After removal of the culture supernatants, the culture plates can be rinsed 3 times with phosphate buffered saline (PBS) and reserved for protein determination by known methods, e.g., Hayner et al. 1982, Tissue Culture Methods 7: 77-80.

The present invention further provides methods for the treatment of liver damage. In this regard, the differentiated hepatocyte-like cells of the invention can be used for the treatment of any disease causing or contributing to liver damage, including but not limited to, amebic liver abscess, autoimmune hepatitis, biliary atresia, cirrhosis, coccidioidomycosis; disseminated, delta agent (Hepatitis D), drug-induced cholestasis, hemochromatosis, Hepatitis A, Hepatitis B, Hepatitis C, hepatocellular carcinoma, liver cancer, liver disease due to alcohol, primary biliary cirrhosis, pyogenic liver abscess, Reye's syndrome, Sclerosing cholangitis and Wilson's disease.

The present invention provides methods for the treatment of liver damage by administering to an individual in need thereof, an effective amount of the differentiated hepatocyte-like cells of the invention. By effective amount is meant an amount sufficient to provide a beneficial effect to the individual receiving the treatment, such as an amount to ameliorate symptoms of liver disease/damage and/or to improve liver function. In certain embodiments, an effective amount is an amount sufficient to regrow functioning liver. Symptoms of liver disease include but are not limited to, jaundice (yellowing of eyes and skin), severe itching, dark urine, mental confusion or coma, vomiting of blood, easy bruising and tendency to bleed, gray or clay-colored stools, and abnormal buildup of fluid in the abdomen.

A “therapeutic” treatment is a treatment administered to a subject who exhibits signs of pathology for the purpose of diminishing or eliminating those signs.

In one embodiment, the present invention provides methods for improving or restoring liver function by administering an effective amount of the differentiated hepatocyte-like cells of the invention. In this regard, hepatocyte-like cells are differentiated using methods as described herein, from human umbilical cord matrix of an individual patient for autologous (in situations where appropriate cells may have been harvested and stored at the time of birth) or allogeneic transplantation to a histocompatible recipient according to the methods described herein. The cells are cultured as described herein, harvested, and may be introduced into the spleen, circulation, and/or peritoneum of a patient suffering from degenerative liver diseases of any origin, secondary to viral infection, toxin ingestion, or inborn metabolic errors, etc. Wherever possible, radiologically guided, minimally invasive methods are used to implant the cells. Cells genetically engineered with genes encoding enzymes designed to improve hepatic function are also contemplated herein.

In one particular embodiment, the hepatocyte-like cells of the present invention are administered to an individual undergoing a liver transplant.

The hepatocyte-like cells of the present invention may be administered either alone, or as a pharmaceutical composition in combination with diluents and/or with other components such as hepatocyte growth factors or other hormones or cell populations. Briefly, compositions of the present invention may comprise a hepatocyte-like cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention may formulated for intravenous or parenteral administration or for administration directly into the liver.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an effective amount”, or “therapeutic amount” is indicated, the precise amount of the compositions of the present invention to be administered can be determined by a physician with consideration of individual differences in age, weight, disease, extent of infection or liver damage, and condition of the patient (subject). In certain embodiments, a pharmaceutical composition comprising the cells described herein may be administered at a dosage of 10³ to 10⁷ cells/kg body weight and in certain embodiments, 10⁵ to 10⁶ cells/kg body weight, including all integer values within those ranges. The hepatocyte-like cell compositions may also be administered multiple times at these dosages. The optimal dosage and treatment regime for a particular patient can readily be determined by one skilled in the art of medicine by monitoring the patient for signs of disease and adjusting the treatment accordingly.

The administration of the subject compositions may be carried out in any convenient manner, including by injection, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the hepatocyte-like cell compositions of the present invention are administered to a patient by intradermal or subcutaneous injection. In another embodiment, the hepatocyte-like cell compositions of the present invention are administered by i.v. injection. The compositions of hepatocyte-like cells may be injected directly into the liver.

In yet another embodiment, the pharmaceutical composition can be delivered in a controlled release system. In one embodiment, a pump may be used (see Langer, 1990, Science 249:1527-1533; Sefton 1987, CRC Crit. Ref. Biomed. Eng. 14:201; Buchwald et al., 1980; Surgery 88:507; Saudek et al., 1989, N. Engl. J. Med. 321:574). In another embodiment, polymeric materials can be used (see Medical Applications of Controlled Release, 1974, Langer and Wise (eds.), CRC Pres., Boca Raton, Fla.; Controlled Drug Bioavailability, Drug Product Design and Performance, 1984, Smolen and Ball (eds.), Wiley, New York; Ranger and Peppas, 1983; J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled release system can be placed in proximity of the therapeutic target, thus requiring only a fraction of the systemic dose (see, e.g., Medical Applications of Controlled Release, 1984, Langer and Wise (eds.), CRC Pres., Boca Raton, Fla., vol. 2, pp. 115-138).

The cell compositions of the present invention may also be administered using any number of matrices. Matrices have been utilized for a number of years within the context of tissue engineering (see, e.g., Principles of Tissue Engineering (Lanza, Langer, and Chick (eds.)), 1997. The present invention utilizes such matrices within the novel context of acting as an artificial liver to support, maintain, or modulate liver function. Accordingly, the present invention can utilize those matrix compositions and formulations which have demonstrated utility in tissue engineering. Accordingly, the type of matrix that may be used in the compositions, devices and methods of the invention is virtually limitless and may include both biological and synthetic matrices. In one particular example, the compositions and devices set forth by U.S. Pat. No. 5,980,889; 5,913,998; 5,902,745; 5,843,069; 5,787,900; or 5,626,561 are utilized. Matrices comprise features commonly associated with being biocompatible when administered to a mammalian host. Matrices may be formed from both natural or synthetic materials. The matrices may be non-biodegradable in instances where it is desirable to leave permanent structures or removable structures in the body of an animal, such as an implant; or biodegradable. The matrices may take the form of sponges, implants, tubes, telfa pads, fibers, hollow fibers, lyophilized components, gels, powders, porous compositions, or nanoparticles. In addition, matrices can be designed to allow for sustained release seeded cells or produced cytokine or other active agent. In certain embodiments, the matrix of the present invention is flexible and elastic, and may be described as a semisolid scaffold that is permeable to substances such as inorganic salts, aqueous fluids and dissolved gaseous agents including oxygen.

A matrix is used herein as an example of a biocompatible substance. However, the current invention is not limited to matrices and thus, wherever the term matrix or matrices appears these terms should be read to include devices and other substances which allow for cellular retention or cellular traversal, are biocompatible, and are capable of allowing traversal of macromolecules either directly through the substance such that the substance itself is a semi-permeable membrane or used in conjunction with a particular semi-permeable substance.

In certain embodiments of the present invention, the hepatocyte-like cell compositions are administered to an individual in conjunction with (e.g. before, simulataneously or following) any number of relevant treatment modalities, including but not limited to treatment with agents such as antiviral agents, chemotherapy, radiation, immunosuppressive agents, such as cyclosporin, azathioprine, methotrexate, and mycophenolate.

In a further embodiment, the cell compositions of the present invention are administered to a patient in conjunction with (e.g. before, simulataneously or following) a liver transplant.

The dosage of the above treatments to be administered to a patient will vary with the precise nature of the condition being treated and the recipient of the treatment. The scaling of dosages for human administration can be performed according to art-accepted practices.

### EXAMPLES

### Example 1

### Hepatic Differentiation of Human Umbilical Cord Matrix Stem Cells

This example describes the differentiation of human umbilical cord matrix stem cells into hepatocytes-like cells.

Umbilical cord matrix cells were isolated from umbilical cords as follows: Umbilical cords were obtained from full term infants in accordance with the University of Kansas Human Subjects Approval. The human umbilical cord matrix (HUCM) cells were grown from umbilical cord tissue that was processed in the following manner: The cord was prepared for processing by rinsing in a 1000 mL beaker containing approx. 500 mL of 95% ethanol or sufficient amount to completely cover the cord, for 30 seconds. The cord was then flamed until the ethanol dissipated, then washed thoroughly 2×, for 5 minutes, in cold sterile PBS (500 mL). Next, the cord was submerged in 500 mL Betadine solution 1× for 5 minutes followed by rinsing thoroughly 2× for 5 minutes with cold sterile PBS (500 mL) to remove the Betadine. The cord was then sectioned into ˜5 cm pieces. When the cord piece was completely dissected and cleaned of blood with PBS, it was placed into the 50 ml tube or 100 mm tissue culture plate containing 40 U/mL hyaluronidase/0.4 mg/mL collagenase solution for 30 minutes in a 37° C. humidified incubator with 5% CO₂. The digested piece of cord section was then placed into a sterilized cell strainer and pestle with a 40 mesh screen installed. The apparatus was then placed on a sterile 100 mm Petri dish, and 5-10 mL of Defined Media (DM) was added which contains: 58% low glucose DMEM (Invitrogen, Carlsbad, Calif.), 40% MCDB201 (Sigma, St. Louis, Mo.), 1× insulin-transferrin-selenium-A (Invitrogen, Carlsbad, Calif.), 0.15 g/mL AlbuMAX I (Invitrogen, Carlsbad, Calif.), 1 nM dexamethasone (Sigma, St. Louis, Mo.), 100 μM ascorbic acid 2-phosphate (Sigma, St. Louis, Mo.), 100 U penicillin, 1000 U streptomycin (Mediatech, Inc., Herdon, Va.), 2% fetal bovine serum (FBS) (Invitrogen, Carlsbad, Calif.), 10 ng/mL epidermal growth factor (EGF) (R & D Systems, Minneapolis, Minn.), and 10 ng/mL platelet-derived growth factor BB (PDGF-BB) (R & D Systems, Minneapolis, Minn.).

The tissue is triturated and pushed through a strainer with a pestle until most of the tissue had lost its structure and the fluid was collected with a 10 mL pipet. The sample was then centrifuged at 750 RCF (×g) for 10 minutes. The media was aspirated off the media being careful not to disturb pellet. The pellet was resuspended in the appropriate volume of DM to obtain the desired range where antimicrobial control was obtained. The diluted cell preparation was then seeded into 6-well plates or other tissue culture vessel as appropriate. The cells were placed in a 37° C. humidified incubator with 5% CO₂ and left undisturbed for ˜24 hours. 24-48 hours after isolation, non-adherent cells were removed by washing three times with sterile PBS. Fresh DM was changed every two days. When culture confluency of between 50-80% was reached the cells were harvested using 0.05% trypsin/0.53 mM EDTA solution and re-plated into a T25 culture flask for further expansion in DM. Cultures were maintained at the stated confluency (50-80%) for propagation. Cultures were maintained in a 37° C. humidified incubator with 5% CO₂ and were replenished with fresh DM every 2-3 days.

The isolated HUMCs were shown to be multipotential and differentiated into osteocytes, chondrocytes, adipocytes and neuronal-like cells. This was shown by photomicrograph. These unique cells were also shown to express stem cell markers cKit, smooth muscle actin, neuron specific enolase (NSE), and neurofilament M (NFM). See also US Patent Application Publication No. US Patent Application Publication No. 20040136967.

The differentiation protocol was a sequential addition of exogenous factors. Prior to induction, cells were seeded on 0.1% gelatin coated T75 culture flasks at a density of 2.0-3.0E06 cells/flask and allowed to adhere overnight. Cells were then treated for two days in pre-induction media consisting of: Serum free IMDM (Invitrogen, Carlsbad, Calif.), 20 ng/ml recombinant human epidermal growth factor (rhEGF) (R & D Systems, Minneapolis, Minn.), 10 ng/ml recombinant human basic fibriblast growth factor (rhbFGF) (Chemicon, Temecula, Calif.), and Pen/Strep. Differentiation was accomplished using a two step process where cells were cultured for 7 days in differentiation media containing: Iscove's Modified Dulbecco's Medium (IMDM), 20 ng/ml recombinant human hepatocyte growth factor (rhHGF) (Chemicon, Temecula, Calif.), 10 ng/ml rhbFGF, 0.61 g/L nicotinamide (Sigma, St. Louis, Mo.), 2% FBS, Pen/Strep. Cells were then cultured in maturation media up to 10 weeks containing: IMDM, 20 ng/ml Human Oncostatin M (Bioscource, Camarillo, Calif.), 1 umol/L dexamethasone, 50 mg/ml ITS+ premix (Sigma, St. Louis, Mo.), 2% FBS, and Pen/Strep. Media was changed every three days and hepatic differentiation was assessed in a temporal manner.

The following methods were used to assess differentiation in the cells:

Immunocytochemistry. Differentiated cells were fixed with 4% paraformaldehyde in PBS for 10 min and then washed in PBS. Cells were permeabilized with 0.2% Triton X-100 in PBS for 5 min, washed and then blocked in 0.2% Triton X-100, 2% normal serum in PBS for 1 h, and then incubated with antibodies to alpha 1 fetoprotein (AFP), cytokeratin 18 (CK18), cytokeratin 19 (CK19), glutamine synthetase (GS), hepatocytes nuclear factor 4 alpha (HNF4α), Nanog, smooth muscle actin (SMA), Von Willebrand Factor (VWF) (1:100, Abcam, Cambridge, Mass.). After washing three times with PBS, cells were incubated with secondary antibody (1:200, Alexa Fluor 488, Molecular Probes, Eugene, Oreg.). Images were obtained with a 510 Zeiss laser scanning microscope under 63× oil-immersion lens, or Nikon Eclipse TE 2000U with Cool SNAPcf (Photometrix) digital camera using MetaMorph imaging software.

RNA isolation and Reverse Transcription Polymerase Chain Reaction (RT-PCR.): RNA was isolated from cells on RNeasy Quick spin columns (Qiagen, Valencia, Calif.) and converted to cDNA using random hexamers and SuperScript II reverse transcriptase (Invitrogen, Carlsbad, Calif.). PCR was performed using a BioRad I-Cycler. A primer list is provided in Table 1 below. Products were resolved by 2% agarose gel electrophoresis and visualized by ethidium bromide staining. Expression of numerous hepatocyte-specific genes was analyzed, including CK18, cytokeratin 18; HNF3-β, hepatocyte nuclear factor 3β; CK19, cytokeratin 19; AFP, alpha fetoprotein; Alb, albumin; and CYP2B6, cytochrome P450 2 family.

TABLE 1

PRIMERS USED FOR RT-PCR

| | | | |
|---|---|---|---|
| | | Pro- | |
| | | duct | SEQ |
| | | size | ID |
| Gene | Sequence | (bp) | NO: |

| | | | |
|---|---|---|---|
| AFP | F 5′-TGC AGC CAA AGT GAA GAG GGA | 216 | 1 |
| | AGA-3′ | | |
| | R 5′-CAT AGC GAG CAG CCC AAA GAA | | 2 |
| | GAA-3′ | | |
| CAR | F 5′-GAC CAG ATC TCC CTT CTC AAG- | 305 | 3 |
| | 3′ | | |
| | R 5′-CTC AGG CTC TTG GAG CTG CAG- | | 4 |
| | 3′ | | |
| CK-19 | F 5′-ATG GCC GAG CAG AAC CGG AA-3′ | 328 | 5 |
| | R 5′-CCA TGA GCC GCT GGT ACT CC-3′ | | 6 |
| CYP2B6 | F 5′-GAC GCT ACG TTT CAG TCT TTC- | 204 | 7 |
| | 3′ | | |
| | R 5′-GCT GAA TAC CAC GCC ATA G-3′ | | 8 |
| CYP3A4 | F 5′-TTC CTA AGG ACT TCT GCT TTG | 333 | 9 |
| | C-3′ | | |
| | R 5′-TGT GGA GGA AAT TAT TGA GAA | | 10 |
| | ATG-3′ | | |
| GAPDH | F 5′-ACC AGT GGA TGC AGG GAT-3′ | 470 | 11 |
| | R 5′-TCA ACG GCA CAG TGA AGG-3′ | | 12 |
| HNF3-β | F 5′-TAT TGG CTG CAG CTA AGC GG-3′ | 508 | 13 |
| | R 5′-GAC TCG GAC TCA GGT GAG GT-3′ | | 14 |
| HNF4-α | F 5′-CCA AGT ACA TCC CAG CTT TC-3′ | 295 | 15 |
| | R 5′-TTG GCA TCT GGG TCA AAG-3′ | | 16 |
| PEPCK | F 5′-TCT GCC AAG GTC ATC CAG G-3′ | 290 | 17 |
| | R 5′-GTT TTG GGG ATG GGC ACT G-3′ | | 18 |
| PGC-1 | F 5′-GGC ACG CAG TCC TAT TCA TT-3′ | 800 | 19 |
| | R 5′-ACA GGG GAG AAT TTC GGT G-3′ | | 20 |
| PPAR-γ | F 5′-AGA CCA CTC CCA CTC CTT TG-3′ | 129 | 21 |
| | R 5′-AGG TCA TAC TTG TAA TCT GC-3′ | | 22 |
| PXR | F 5′-CAA GCG GAA GAA AAG TGA ACG- | 442 | 23 |
| | 3′ | | |
| | R 5′-CTG GTC CTC GAT GGG CAA GTC- | | 24 |
| | 3′ | | |
| β-actin | F 5′-TGA ACT GGC TGA CTG CTG TG-3′ | 174 | 25 |
| | R 5′-CAT CCT TGG CCT CAG CAT AG-3′ | | 26 |

Cellular uptake of Indocyanine Green (ICG.): ICG was dissolved to an initial concentration of 5 mg/mL in solvent. The solution was then diluted to 1 mg/mL in maturation media and added to the culture dish and incubated at 37° C. in a humidified incubator at 5% CO₂ for 10-15 minutes. The cells were washed thoroughly with sterile PBS and then visualized under a light microscope. After examination, the PBS was then removed and maturation media was added and the cells incubated at 37° C. in a humidified incubator at 5% CO₂ for ˜4-6 hours to confirm elimination of ICG.

Cellular uptake of Low-Density Lipoprotein (LDL.): Dil-Ac-LDL was diluted in maturation media to 10 μg/mL, added to cells, and incubated for 4 hours at 37° C. in a humidified incubator. After incubation, media was removed containing the Dil-Ac-LDL and the cells were washed 2× with probe-free maturation media. Cells were visualized using standard rhodamine excitation: Cells were compared to positive and negative cultures for comparison purposes.

Periodic Acid-Schiff (PAS) Staining and Diastase Treatment: Cells were washed 2× with PBS and fixed with 4% paraformaldehyde for 10 minutes, washed 1×PBS, and permeabilized with 0.1% Triton-X100 dissolved in PBS for 5 minutes. Cells were incubated with 0.2 g/40 mL diastase at 37° C. for 1 hr for glycogen digestion. Cells were then oxidized in 1% periodic acid for 5 minutes; rinsed 3× with PBS, then treated with Schiff's reagent for 15 minutes and rinsed 3× with PBS. Cells were counter-stained with H&E for 1 minutes and washed thoroughly with PBS. Samples were imaged under a light microscope.

Immunoblotting: Cells were washed 2× with ice-cold PBS (Cellgro, Dulbecco's Phosphate Buffered Salt Solution w/o magnesium and calcium) Tissue culture plates were subjected to ice-cold lysis buffer (Sigma, CelLytic™-MT Mammalian Tissue Lysis/Extraction Reagent, C-3228) and protease inhibitor cocktail (Sigma, Protease Inhibitor Cocktail, P-8340). Cells were removed from tissue culture flasks by scraping and transferred to a microfuge tube. Cells were then passed through a 27 gauge needle, and then centrifuged at 14,000 rpm in microfuge for 10 minutes at 4° C. Supernatant was assayed for protein with BCA method. To the supernatant, 4× sample buffer was added and incubated at 85° C. for 30 minutes. Lysates were separated on 4-20% SDS-polyacrylamide gel (Pierce, 4-20% Precise™ Protein Gels, 25244) and transferred to PVDF (Pierce, 88518.) For western blotting: AFP, Albumin, CK18, CK19, SMA (Abcam, Cambridge, Mass.), horseradish peroxidase conjugated rabbit anti-goat (Invitrogen, 81-1620), or goat anti-rabbit (Invitrogen, 62-6120) was used at 1:20,000 for detection with the Super Signal West Pico chemilluminescence system (Pierce, 34077.)

Phenobarbital, Rifampicin, Forskolin, and 8-Br-cAMP Treatment of Differentiated Cells Differentiated cells were trypsinized and seeded on 6-well plates at a seeding density of 10,000 to 20,000 cells/cm² using maturation media and allowed to adhere overnight. Cytochromes were induced by treatment with; Rifamicin (RIF), 20 um; Penobarbital (PB), 2 mM; forskolin, 50 uM; 8-Bromo-cAMP, 1 mM (Tocris, Ellisville, Mo.); and vehicle controls for 24-hour period. mRNA was then harvested and then analyzed by RT-PCR.

Flow cytometry: HUCM cells at 1×106 cells/mL were fixed with methanol at 4° C. for 5 min and blocked with PBS and 5% bovine serum albumin at 4° C. for 1 h. Cells were incubated with 1 μg/mL primary antibodies at 4° C. for 1 h. Cells were washed three times with PBS and then incubated with appropriate secondary FITC conjugates (1:100, goat anti-mouse, donkey anti-goat, goat anti-rabbit, Molecular Probes, Eugene, Oreg.) for 30 min on ice. Cells were washed twice in PBS and analyzed using a FACSCalibur flow cytometer (Beckman Coulter, Miami, Fla.). Ten thousand cells (no gating) were collected and analyzed in the FL1 channel. All analyses were based on control cells (incubated with either isotype specific IgG or respective secondary conjugates alone) to establish the background signal.

Results:

After 4 weeks in hepatogenic media, the UCM cells were shown by immunofluorescent staining to express albumin and αFP as compared to control UCM cells cultured in control media. HUMCs grown in control media had no increased expression of albumin from two to four weeks. Differentiated cells expressed higher albumin production compared to undifferentiated cells at two weeks, and even more so expression at four weeks post-induction aFP was not present in undifferentiated HUMCs. After four weeks, differentiated cells showed αFP production in the perinuclear region. Smooth muscle actin (SMA) was well structured in undifferentiated HUMCs. At four weeks, SMA was more disorganized in the hepatic induced cells. Induced HUMCs also developed a more polygonal shape, similar to hepatocellular cells, and lost the spindle morphology of undifferentiated stem cells.

HUMCs undergo morphological changes under hepatogenic conditions: HUMC typically underwent morphological changes during the differentiation protocol. These changes were tracked to assess the efficacy of the different growth factors that were applied. Cells were typically bi-nucleated bipolar myofibroblasts that did not form colonies or clusters before pre-induction. When cells were cultured in pre-induction media, cell proliferation halted, but maintained their general morphology. After induction and maturation, cells were mostly mononuclear and heterogeneous with high nucleus to cytoplasmic ratio. Differentiated cells were more polygonal to cuboidal shape and displayed lipid droplet inclusions. Cells did not pile up but did form cannicular type structures that could be observed without a microscope. Phase-contrast (DIC) photomicrograph of differentiated cells showed morphological changes of HUCM cells. The differentiated hepatocyte-like cells under hepatogenic differentiation conditions developed what appeared as sinusoids at 4 weeks post-induction.

Functional analysis of differentiated HUCM cells (Glycogen, ICG, and LDL-uptake): HUMC derived hepatocyte-like cells acquire functional properties (glycogen production.) Glycogen is a simple intracytoplasmic polysaccharide found in abundance in the liver cells. To demonstrate glycogen storage, differentiated cells were stained with PAS. Positive staining for glycogen was shown in differentiated cells but not in undifferentiated cells suggesting the capacity of glycogen storage found in liver parenchymal cells. (Demonstration of glycogen by PAS staining was found in differentiated cells but not shown in undifferentiated cells.) Glycogen can be digested by diastase in cell culture conditions. To demonstrate positive glycogen staining differentiated cells were pretreated with Diastase solution and no positive staining for glycogen was observed.

Cellular uptake of anionic dye, ICG, was examined in differentiated and undifferentiated HUMCs to determine hepatic function. ICG-positive cells were not observed in undifferentiated cells. ICG staining was observed in differentiated cells as early as 1 week with the greatest amount of positive staining later. At 1 mg/mL ICG concentration, no adverse effects were observed. As a control, cell line Hep G2 was used, and observed to have positive ICG staining. ICG was cleared from cells after re-application of maturation media.

Liver cells express LDL receptors for regulation of cholesterol homeostasis in mammals. To determine if differentiated cells exhibited cellular uptake of LDL, cells were treated with Dil-Ac-LDL. The differentiated cells exhibited lower levels of staining when sampled early in the post-induction phase than in late post-induction where LDL incorporation was further increased.

Immunoblotting and RT-PCR analysis of induced HUCM cells reveal temporal expression pattern (profile) of hepatocyte-specific genes and proteins: Protein expression levels of CK18 and alfa-fetoprotein remained about the same during the differentiation course where albumin increased at two to four weeks post-induction. CK19 decreased in expression by two weeks post-induction.

RT-PCR analysis showed detected alpha-fetoprotein throughout the differentiation course. HNF3β was detected as early as one week post-induction. CYP2B6 expression was detected as late as four weeks post-induction and CK-19 decreased after two weeks post induction. These results indicate the maturating of hepatocyte-like cells, where the appearance of early to late markers is seen, which is consistent with a differentiating cell.

RT-PCR analysis of the expression of inducible markers four weeks post-induction: Differentiated cells that were treated with either phenobarbital (PB), rifampicin (RIF), 8-Bromoadenosine-3′,5′-Cyclic Adenosine Monophosphate (8-Br-cAMP) or forskolin showed a number of hepatocyte-inducible genes or an increase in expression levels. Constitutive androstane receptor (CAR), pregnane X receptor (PXR), peroxisome proliferators-activated receptor γ coactivator-1α (PGC-1) coordinately regulate enzymes in drug metabolism and gluconeogenesis. Phosphoenolpyruvate carboxykinase (PEPCK) and peroxisome proliferators-activated receptor-γ (PPAR-γ), are key gluconeogenic enzymes. CYP3A4 a cytochrome P450 (CYP) Phase I monooxygenase system enzyme important for endo- and xenobiotic metabolism. Hepatocyte nuclear factor 4α (HNF4α) is a master transcription regulator for lipid and glucose metabolic pathways. These genes either showed elevated expression in the differentiated hepatocyte-like cells or were induced in these cells upon treatment with PB, RIF, 8-Br-cAMP or forskolin. The differentiated cells expressed these hepatocyte-specific genes in a time-dependent manner. Furthermore, these markers have not been previously shown to be expressed in cells differentiated into the hepatocyte lineage from other types of stem cells (see e.g., Lee O K, et al. Blood. 2004; 103(5):1669-1675; Yamada T, et al., Stem Cells. 2002; 20(2):146-154; Wang et al., Liver Transpl. 2005 June; 11(6):635-43; Hong S H, et al. Biochemical and Biophysical Research Communications. 2005; 330(4):1153-1161).

Immunocytochemical staining verify hepatic differentiation: To confirm expression of hepatogenic markers we examined the differentiated HUMCs by immunocytochemical staining. Cells were grown on 8-well chamber slides, fixed and stained with poly- or monoclonal antibodies against CK18, cytokeratin 18; HNF4-α, hepatocyte nuclear factor 4α; CK19, cytokeratin 19; AFP, alpha fetoprotein; GS, glutamine synthetase; VWF, Von Willebrand Factor; Nanog; SMA, smooth muscle actin, and Alexa Fluor 488 secondary antibodies. Cell nucleus was stained with TO-PRO-3 and imaged with an Zeiss confocal microscope at 40× power. Immunofluorescence analysis showed that differentiated cells stain for; CK18, HNF4α, AFP, GS, vWF, and negative staining for CK19 and Nanog. SMA still persists in differentiated cells but at a lower level than undifferentiated. Magnified view of the nucleus showed localization of HNF4α. These results indicated that the hepatogenic markers increased and correlate with protein and mRNA expression.

Cytochromes are differentially expressed during HUCM cell differentiation: 2 mM PB treatment at four weeks differentiation induced PXR, HNF4α, and CYP3A4. Expression levels of CAR and PGC-1 increased and PPAR-γ stayed the same. 25 μM RIF treatment induced PEPCK, PXR, HNF4α, and CYP3A4.

Thus, this example demonstrates that UCM cells cutured as described herein differentiated into cells showing specific hepatocyte characteristics including morphological, phenotypical and functional hepatocyte-like characteristics.

### Example 2

### Hepatic Differentiation of Human Umbilical Cord Matrix Stem Cells Using Hepatocyte Feeder Cell Layer

This example shows the hepatic differentiation of HUCM cells following coculture on a feeder layer comprised of heat-shocked HB8065 cells, a hepatocellular carcinoma cell line.

UCM cells were isolated from umbilical cords as previously described (see e.g., US Patent Application Publication No. 20040136967). HUCM cells were seeded on a porous membrane in a transwell insert. The transwell insert created in the culture well an upper compartment, a microporous membrane (on the insert) and a lower compartment. The HUCM cells were seeded on the porous membrane in DMEM, 2% FBS and with the heat-shocked HB8065 hepatocyte feeder layer in the lower compartment. Control HUCM cells were cultured in DMEM with 2% FBS only. Differentiation was assessed by immunofluorescence, RT-PCR and protein chemistry.

Coculture of HUCM with a hepatocyte feeder layer increased the presence of hepatocyte specific proteins (albumin and aFP) and led to more disorganized expression of SMA.

Results from PCR show that albumin was strongly expressed in the hepatocellular carcinoma cell line used as the feeder layer, and weakly expressed in undifferentiated HUCM cells as well as in the differentiation control. This correlates with immunocytochemistry results, where albumin was detected at low levels in undifferentiated cells. This gene continued to be expressed throughout the differentiation experiment, and showed signs of slight increased intensity, especially at 4 weeks post-induction. Beta-actin was used as a positive control for PCR, and was present in all cells.

Thus, the HB8065 cell line produces factors sufficient to induce hepatic differentiation of HUCM cells.

All of the above U.S. patents, U.S. patent application publications, U.S. patent applications, foreign patents, foreign patent applications and non-patent publications referred to in this specification and/or listed in the Application Data Sheet, including but not limited to U.S. Provisional Patent Application No. 60/817,251, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A method for differentiating umbilical cord matrix cells into hepatocyte-like cells, comprising:
a. contacting umbilical cord matrix cells with Pre-induction Media;
b. contacting umbilical cord matrix cells with Differentiation Media; and
c. contacting umbilical cord matrix cells with Maturation Media;
for a time sufficient to differentiate the umbilical cord matrix cells into hepatocyte-like cells.

2. A method for evaluating the toxicity of a compound in vitro, comprising
a. contacting a hepatocyte-like cell differentiated from umbilical cord matrix cells according to claim 1 with said compound; and
b. measuring the viability of said hepatocyte-like cell, wherein a decrease in viability in the presence of said compound compared to that in the absence of said compound indicates that said compound is toxic in vivo.

3. A method for evaluating the activity of a compound in vitro, comprising
a. contacting a metabolically active hepatocyte-like cell differentiated from umbilical cord matrix cells according to claim 1 with said compound; and
b. measuring the metabolic activity of said hepatocyte-like cell, wherein a decrease or increase in metabolic activity in the presence of said compound compared to that in the absence of said compound indicates that said compound has activity in vivo.

4. A method for evaluating the activity of a compound in vitro, comprising
a. contacting a first metabolically active hepatocyte-like cell differentiated from umbilical cord matrix cells according to claim 1 with said compound to generate a cell supernatant; and
b. contacting a second metabolically active hepatocyte-like cell differentiated from umbilical cord matrix cells according to claim 1 with said supernatant; and
c. measuring the metabolic activity of said second hepatocyte-like cell, wherein a decrease or increase in metabolic activity in the presence of said supernatant compared to that in the absence of said supernatant indicates that said compound has activity in vivo.

5. A method for evaluating the toxicity of a compound in vitro, comprising
a. contacting a first metabolically-active hepatocyte-like cell differentiated from umbilical cord matrix cells according to claim 1 with said compound to generate a cell supernatant;
b. contacting a second metabolically-active hepatocyte-like cell differentiated from umbilical cord matrix cells according to claim 1 with said cell supernatant; and
c. measuring the viability of said second hepatocyte-like cell, wherein a decrease in viability in the presence of said supernatant compared to that in the absence of said supernatant indicates that said compound is toxic in vivo.

6. A method for evaluating the activity of a compound in vitro, comprising
a. contacting a hepatocyte-like cell differentiated from umbilical cord matrix cells according to claim 1 with said compound; and
b. measuring the expression of a cytochrome P450 gene in the hepatocyte-like cell, wherein an increase or decrease in expression of the cytochrome P450 gene in the presence of said compound compared to that in the absence of said compound indicates that said compound has actvity in vivo.

7. A method for evaluating the activity of a compound in vitro, comprising
a. contacting a first metabolically active hepatocyte-like cell differentiated from umbilical cord matrix cells according to claim 1 with said compound to generate a cell supernatant; and
b. contacting a second metabolically active hepatocyte-like cell differentiated from umbilical cord matrix cells according to claim 1 with said supernatant; and
c. measuring expression of a cytochrome P450 gene in said second hepatocyte-like cell, wherein an increase or decrease in expression of the cytochrome P450 gene in the presence of said supernatant compared to that in the absence of said supernatant indicates that said compound has activity in vivo.

8. The method of claim 6 or claim 7 wherein the cytochrome P450 gene expression is measured using the polymerase chain reaction.

9. The method of claim 6 or claim 7 wherein the cytochrome P450 gene expression is measured by measuring enzyme activity.

10. A method for determining drug interactions, comprising:
contacting a first hepatocyte-like cell differentiated from umbilical cord matrix cells according to claim 1 with a first compound;
contacting a second hepatocyte-like cell differentiated from umbilical cord matrix cells according to claim 1 with a second compound;
contacting a third hepatocyte-like cell differentiated from umbilical cord matrix cells according to claim 1 with the first and the second compound;
measuring the metabolic activity of the first, second and third hepatocyte-like cell, wherein a decrease or increase in metabolic activity in the third hepatocyte-like cell as compared to the first or the second hepatocyte-like cell or both indicates a drug interaction.

11. A method for determining drug interactions, comprising:
contacting a first hepatocyte-like cell differentiated from umbilical cord matrix cells according to claim 1 with a first compound;
contacting a second hepatocyte-like cell differentiated from umbilical cord matrix cells according to claim 1 with a second compound;
contacting a third hepatocyte-like cell differentiated from umbilical cord matrix cells according to claim 1 with the first and the second compound;
measuring the viability of the first, second and third hepatocyte-like cells, wherein a decrease or increase in viability in the third hepatocyte-like cell as compared to the first or the second hepatocyte-like cell or both indicates a drug interaction.

12. A method for determining drug interactions, comprising:
contacting a first hepatocyte-like cell differentiated from umbilical cord matrix cells according to claim 1 with a first compound;
contacting a second hepatocyte-like cell differentiated from umbilical cord matrix cells according to claim 1 with a second compound;
contacting a third hepatocyte-like cell differentiated from umbilical cord matrix cells according to claim 1 with the first and the second compound;
measuring the expression of a cytochrome P450 gene in the first, second and third hepatocyte-like cells, wherein a decrease or increase in the expression of a cytochrome P450 gene in the third hepatocyte-like cell as compared to the first or the second hepatocyte-like cell or both indicates a drug interaction.

13. A method for improving or restoring liver function in an individual in need thereof comprising administering to the individual in need thereof a population of hepatocyte-like cells differentiated from umbilical cord matrix cells according to claim 1.

14. A method for treating cirrhosis of the liver in an individual in need thereof comprising administering to the individual a population of hepatocyte-like cells differentiated from umbilical cord matrix cells according to claim 1.

15. A method for treating liver damage comprising administering to an individual who has sustained liver damage a population of hepatocyte-like cells differentiated from umbilical cord matrix cells according to claim 1.

16. A method for treating hepatitis comprising administering to an individual who has sustained liver damage a population of hepatocyte-like cells differentiated from umbilical cord matrix cells according to claim 1.

17. A panel of umbilical cord matrix-derived hepatocyte-like cells comprising at least two umbilical cord matrix-derived hepatocyte-like cells wherein the at least two umbilical cord matrix-derived hepatocyte-like cells are derived from different subjects, and wherein the umbilical cord matrix-derived hepatocyte-like cells are separate one from the other.

18. The panel of claim 17 wherein the different subjects are genetically different.

19. The panel of claim 17 wherein the different subjects are of different sexes.

20. The panel of claim 17 wherein the at least two umbilical cord matrix-derived hepatocyte-like cells are separated one from the other in a multi-well plate.

21. The panel of claim 17 wherein the panel comprises at least three different umbilical cord matrix-derived hepatocyte-like cells.

22. The panel of claim 17 wherein the panel comprises at least four different umbilical cord matrix-derived hepatocyte-like cells.

23. The panel of claim 17 wherein the panel comprises between 5 and 100 different umbilical cord matrix-derived hepatocyte-like cells.

24. A drug screening kit comprising a panel of claim 17 and at least one reagent for measuring at least one cytochrome P450 enzyme activity or gene expression.

25. A drug screening kit of claim 24 further comprising at least one medium for culturing the umbilical cord matrix-derived hepatocyte-like cells.

26. A method for differentiating umbilical cord matrix cells into hepatocyte-like cells, comprising:
a. seeding umbilical cord matrix cells on a 0.1% gelatin coated tissue culture plate;
b. contacting umbilical cord matrix cells with a Pre-induction Media comprising 10-30 ng/ml recombinant human epidermal growth factor and 5-15 ng/ml recombinant human basic fibriblast growth factor;
c. contacting umbilical cord matrix cells with a Differentiation Media comprising 10-30 ng/ml recombinant human hepatocyte growth factor, 5-15 ng/ml rhbFGF and 0.5-1.0 g/L nicotinamide; and
d. contacting umbilical cord matrix cells with a Maturation Media comprising 10-30 ng/ml Human Oncostatin M, 0.5-1.5 umol/L dexamethasone and 30-70 mg/ml ITS+ premix;
for a time sufficient to differentiate the umbilical cord matrix cells into hepatocyte-like cells.
